(19) United States
(12) Patent Application Publication
(10) Pub. No.: US 2020/0368288 A1
(43) Pub. Date: Nov. 26, 2020

(54) Hierarchic Neural Microphysiological System for Brain Function and Disorders

(71) Applicant: The Trustees of Columbia University in the City of New York, New York, NY (US)

(72) Inventors: Raju TOMER, Tenafly, NJ (US); Maria de los Angeles LOZANO, New York, NY (US)

(73) Assignee: The Trustees of Columbia University in the City of New York, New York, NY (US)

(21) Appl. No.: 16/992,316

(22) Filed: Aug. 13, 2020

Related U.S. Application Data
(63) Continuation-in-part of application No. PCT/US2019/017998, filed on Feb. 14, 2019.
(60) Provisional application No. 62/630,577, filed on Feb. 14, 2018.

(51) Int. Cl. A61K 35/30; G01N 33/50; C12N 5/0797

(52) U.S. Cl. CPC A61K 35/30; C12N 5/0623; G01N 33/5008

(57) ABSTRACT

A network of neurospheres (NNet) mimicking the small-world hierarchic-modular architecture of mammalian brains, created by synthetically building a network of inter-connected individual brain microphysiological systems (MPSs).

### CROSS-REFERENCE TO RELATED APPLICATIONS

This application is a continuation-in-part of International Application No. PCT/US2019/017998, filed Feb. 14, 2019, which claims the benefit of U.S. Provisional Application 62/630,577, filed Feb. 14, 2018, each of which is incorporated herein by reference in its entirety.

### FIELD OF INVENTION

This disclosure relates to tissue engineering, more particularly cell culture devices, i.e., organs-on-chips, organoids and neurospheres.

### BACKGROUND

Techniques for creating organs-on-chips are known. Thus, a person of ordinary skill in the art understands how to create three-dimensional cell cultures on a small “chip” to simulate biological characteristics of living organs. The techniques employ those that have been developed for lab-on-chip and cell cultures as well as tissue engineering. Specifically, brain organoids have been created by culturing stem cells in a bioreactor to study disease and brain function and development. For example, brain organoids were described by Lancaster and Knoblich in 2014 (Science. 2014 Jul. 18; 345(6194):1247125. doi: 10.1126/science.1247125. Epub 2014 Jul. 17.). Aggregates of neurons have been observed, for example, See Segev et al Phys Rev Lett. 2003; 90(16):168101. doi: 10.1103/PhysRevLett.90.168101. PubMed PMID: 12732015.

The current state-of-the-art brain microphysiological system (MPS) approaches (“brain-in-a-dish”), including brain organoids, are still not capable of capturing the inherent functional complexities of brain, and its complex disorders. To address these short-comings, the disclosed subject matter provides a novel hierarchic neural MPS approach, i.e., a network of neurospheres (NNet).

### SUMMARY

This disclosure relates to a neurosphere network that contains a plurality of artificial neurospheres on a surface, wherein the neurospheres are interconnected by axons and optionally comprise a mean for measuring neuronal activity and developmental processes. The neurosphere network may be 2D or 3D.

This disclosure also relates to a method of making a neurosphere network by applying neuronal cells to a non-adhesive surface of polysaccharide, such as agarose, or of a silicone-based organic polymer, wherein the neuronal cells on the non-adhesive surface are in cell groups spaced apart from each other, and growing the neuronal cells under culture conditions to form the cell groups into neurospheres, and such that axons form inter-connecting the spaced-apart cell groups or neurospheres, to form a neurosphere network. The cell groups are thus self-aggregated assemblies.

In an embodiment, the neuronal cells are dissociated from mammalian embryonic hippocampus/cortex, or are human iPSC-derived neuroprogenitors cells (NPCs).

The neurospheres may be derived from neuronal cells that are of the same cell type or that are of different cell types. The resulting neurosphere network may contain interconnected neurospheres that represent different tissue types, e.g., corresponding to different parts of the brain.

Optionally, the individual neurospheres are collected and re-positioned strategically on the adhesive surface. The inter-connections may form passively or actively by using guidance cues like Netrins (e.g., using soaked microbeads).

Neurospheres may be spaced apart from each other by substantially the same distance or by varying distances. The distance(s) may vary from μm to mm. In embodiments, the distances may be 1 mm, in further embodiments, the distance may be at least 2 mm. In embodiments, the distances may be at least 5 mm. In embodiments, the distances may be at least 10 mm. In embodiments, the distances may be at least 15 mm. In embodiments, the distances may be a maximum of 1 mm, in further embodiments, the distance may be a maximum of 2 mm. In embodiments, the distances may be a maximum of 5 mm. In embodiments, the distances may be a maximum of 10 mm. In embodiments, the distances may be a maximum of 15 mm. In an embodiment, the distance(s) are determined by self-organization, and in other embodiment by the strategic positioning of the individual neurospheres on the adhesive surface.

The neuronal cells may contain a means for measuring neuronal activity, such as a viral vector, a calcium sensitive dye or protein, or an array of electrodes.

In an embodiment, the non-adhesive surface is a plurality of microwells or is a flat mold. The non-adhesive surface may be a polysaccharide, such as agarose. Alternatively, the non-adhesive surface may be a silicone-based organic polymer, such as polydimethylsiloxane (PDMS). Preferably, the non-adhesive surface is in the form of agarose-based microwells or a flat PDMS mold that are casted in 3D printed stencil mold.

In embodiments, the neurospheres are collected after at least 10 hours after plating, or optionally 12, 14, 16, 24 or more hours after plating to establish the axon inter-connections between neurospheres. In self-organized embodiments, neurospheres axon inter-connections are spontaneously formed.

In embodiments, NNet mimics the small-world hierarchic-modular architecture of mammalian brains (i.e. highly intra-connected modules with fewer inter-modular connections). NNet are created by synthetically building a network of inter-connected individual brain MPSs.

A method includes growing cells (either dissociated from wild-type or diseased mammalian embryonic hippocampus/cortex, or human iPSC-derived neuroprogenitors cells (NPCs)) on a non-adhesive surface made of polydimethylsiloxane (PDMS), agarose or similar materials (see FIGS. 1 and 2) that facilitates the adhesion of cells to each other rather than to the surface. As a result, the cells self-aggregate into 3D assemblies (Neurospheres) formed by all neuronal cell-types, which are then inter-connected either passively or actively by axons forming networks of Neurospheres.

In embodiments, the NNet is used to screen drugs by applying a drug of interest to the NNet and measuring the response as neuronal activity code readout over a period of time. In embodiments, the NNet is used to generate quantitative data indicating fundamental brain computation for example by showing magnitude of brain activity by region or under the effect of selected drugs. In embodiments, the NNet is used to observe and quantify the sizes, positions, and activity level of neuronal ensembles against optical or pharmaceutical perturbations. For personalized medicine, a NNet may be grown from iPS cells derived from a specific patient or patient sub-population, and the resulting NNet tested for drug response.

In an embodiment, this disclosure is a method for screening a compound for activity against a brain disorder, by (a) contacting the compound with a first neural network according to claim 1 or obtained by a method according to any one of claims 2 to 22, wherein the first neural network originated from brain cells obtained from a first mammal or derived from stem cells or iPSC cells of a first mammal presenting a disorder or a biomarker indicative of the brain disorder, (b) contacting the compound with a second neural network according to claim 1 or obtained by a method according to any one of claims 2 to 22, wherein the second neural network originated from brain cells, or brain cells derived from stem cells or iPSC cells, of a second mammal of the same species as the first mammal but lacking the brain disorder or biomarker; and (c) recording activity patterns, using calcium imaging or electrophysiology, over time from the first and second neural networks after the contacting steps, and analyzing the activity patterns to determine a difference in generated signal characteristic of the brain disorder. This method may also include a subsequent step of (d) fixating the neural networks after live recording, and staining and visualizing the molecular identity of the cells for a biomarker.

### BRIEF DESCRIPTION OF THE FIGURES

FIG. 1 shows a hierarchic-modular neurosphere network (NNet) for recapitulating the brain complexities.

FIG. 2 illustrates correlated neuronal activity (ensembles) in individual neurosphere vs. NNet preparations.

FIG. 3 shows preparation NNet of specific configuration.

FIG. 4 illustrates a chronic ketamine treatment NNet model of schizophrenia pathophysiology.

FIG. 5 shows loss of correlation (neuronal ensembles) in chronic ketamine treatment NNet model of schizophrenia pathophysiology.

FIG. 6 illustrates modeling epileptic seizures with NNet.

FIG. 7 shows an example of live calcium imaging data that is collected, analyzed to extract position and activities of individual brain cells.

FIG. 8 shows activity traces of cells identified in FIG. 7.

FIG. 9 plots the synchronization of activities of brain cells (quantified as average pairwise correlation).

FIG. 10 to FIG. 14 show example activity patterns at different days (DIVs).

FIG. 15 shows use of a custom miniaturized microscope, placed directly in the incubator for long-term activity recording, and also real-time data compression and analysis.

FIG. 16 is an example of a fixed sample and its staining to visualize molecular and structural details. These are correlated with the recordings from this sample.

FIG. 17 is a comparison of synchrony of a control and SETD1 mutant (model of Schizophrenia).

FIG. 18 to FIG. 21 show activity pattern images of diseased (in top row in FIG. 18, left column in FIGS. 19 to 21) and control (in bottom row in FIG. 18, right columns in FIGS. 19 to 21).

FIG. 22, FIG. 23, and FIG. 24 shows activity patterns in developing brains as reported in literature (cited in FIGS.).

FIG. 25 to FIG. 33 show results of evaluating the presence of calcium spikes, SPAs, and GDPs in NNets.

FIGS. 34A-34E show various aspects of the Molecular Neuronal Network (MoNNet) approach. (FIG. 34A) Overview of MoNNet preparation and data analysis. Embryonic (E17-18) hippocampus were extracted, and infected with AAV1.Syn.GCaMP6f.WPRE.SV40. The cells were plated on a PDMS mold for self-organized assembly of MoNNet, or on an agarose mold to generate spheroids. System-wide cellular-resolution Ca2+ imaging (30 Hz) was performed, 23 followed by analysis. (FIG. 34B) Comparison of local (i.e. within same spheroids) and global (i.e. across spheroids) functional connectivity in spheroids vs. MoNNet. Left to right: representative images; pairwise(PW) correlation matrix of aggregated activities of spheroids; neuronal PW correlation histograms; density plot for correlation vs. distance. Also see FIG. 39. (FIG. 34C) Left-to-right: spiking events per minute; full width at 75% of peak of dF/F. (FIG. 34D) Progression of MoNNet properties assessed by imaging of 281 MoNNets. Left-to-right: average PW correlation of local(green), global(blue) and all(orange) neurons; histogram comparing the means of three phases; local and global network efficiency of weighted functional graphs; number of detected modules; modularity measure Q. Also see FIG. 40. (FIG. 34E) Representative examples of hierarchical modular organization of MoNNets. Left-to-right: MoNNet functional graph with nodes in same modules colored same, and co-classification probability heatmap and consensus clustering dendrograms. Significance (t-test) markers defined as: *p<0.05, **p<0.01, ***p<0.001, ****p<0.0001. For all plots, error bars are std. dev. For histogram in FIG. 34D, error bars are 95% confidence interval. All scale bars are 500 μm.

FIGS. 35A-35B show pharmacological characterization of MoNNet neuronal and network activity. (FIG. 35A) Before and after treatment comparison of average pairwise correlation, functional graph global efficiency, average firing rate per minute and full width at 75% of maximum dF/F peak. MoNNets in phases I and II were used for treatment. Left subpanel shows effects of GABAergic and Glutamatergic synaptic inhibitors (i.e. Bicuculine [10 μM], D-APV [40 μM] and NBQX [10 μM]), and right shows effects of ionic channel blockers. (i. e. Mefloquine [25 μM], Nifedipine [10 μM] and TTX [1 μM]). Bar heights are mean, and error bars are 95% confidence interval (FIG. 35B) Schematic summary of results presented in A. Statistical significance (paired t-test) is defined as: *p<0.05, **p<0.01, ***p<0.001,****p<0.0001. Also see FIG. 41 and FIG. 42 for controls, representative examples of activity raster plots and correlation graphs and data summary table.

FIGS. 36A-36C show characterizing the cellular architecture of MoNNet. (FIG. 36A) Inmunostaining of 2 weeks old individual spheroids vibratome sections (50 μm thick) to visualize their cellular architecture. Glutaminase and NeuN immunolabeling revealed preferentially peripheral localization of glutamartergic cells, whereas the GAD65 and GFAP expression is preferentially localized in the inner regions. A schematic summary is shown on right. (FIG. 36B) Whole-mount inmunostaining of four weeks old MoNNets. Top row: maxima projections showing Tuj 1, Syn-GCaMP6f and GFAP expression. Bottom row: a confocal optical section showing co- or exclusive (red arrow) labelling of inter-connections with GAD65 or synGCaMP6f signals, suggesting existence of excitatory as well as inhibitory signaling transmission across spheroids unit in MoNNets. (FIG. 36C) Electrophysiology recordings validating the existence of mono-synaptic functional glutamatergic and GABAergic signal transmission across spheroids. For all recordings, a neighboring spheroid, located ˜250-300 μm, away was stimulated at 10 Hz. Top-to-bottom: representative is trace of a cell held at −70 mV showing robust evoked excitatory synaptic transmission across spheroids; representative is trace of a cell held at 0 mV showing robust evoked inhibitory synaptic transmission across spheroids; representative is trace of a cell held at 0 mV showing robust evoked inhibitory synaptic transmission during CNQX (10 μM) blockade of glutamatergic transmission. Scale bars are 100 μm. Also see FIG. 42.

FIGS. 37A-37B show characterization of in vitro models of SCZ-associated network pathophysiology. (FIG. 37A) Comparison of various local and global quantitative measures of MoNNets derived from Setd1a+/− (orange), WT siblings(blue) and Df(16)A+/− (green): average pairwise correlation, global efficiency of functional weighted graphs, number of detected modules, activity data variance captured by the first PCA dimension, predicted spiking events rate per minute; full width at 75% of the maximum peak of ∂F/F traces. Statistical significance was calculated by comparing pooled data (black bars) of Setd1a+/− vs. WT siblings. Statistical significance (t-test) is defined as: *p<0.05, **p<0.01, ***p<0.001, ****p<0.0001. (FIG. 37B) Representative examples of comparing hierarchical modular organization. Nodes belonging to same module are colored same. Co-classification matrix heatmap and clustering dendograms are shown to visualize hierarchical relationship. For all plots, mean and std. deviation are plotted. Also see FIG. 43.

FIGS. 38A-38B show partial rescue of Setd1a+/− MoNNet network pathophysiology by pharmacological inhibition of LSD1 demethylase activity. (FIG. 38A) Histograms comparing various quantitative measures of network function of WT sibling MoNNets (blue) and Setd1a+/− MoNNets treated with DMSO(orange), ORY-1001(green) and TCP(red). All treatments were for two days. From left to right: local and global functional connections stability as measured by the correlation in edge weights of functional graph before and after the treatment; local and global coefficient of determination (r-square fit edge weights before and after treatment) quantifying the predictability of the after treatment state; average pairwise correlation increment as measured by increase in correlation of same pair before and after, averaged over an entire MoNNet; global graph efficiency increment measures of functional graphs; firing rate per minute (FRPM). Statistical significance (t-test) is defined as: *p<0.05, **p<0.01, ***p<0.001, ****p<0.0001 (FIG. 38B) Representative examples of WT sibling MoNNets and Setd1a+1-MoNNets treated with DMSO, ORY-1001 and TCP. Nodes belonging to same modules are colored same. Co-classification matrix heatmap and clustering dendograms are shown to visualize the hierarchical relationship in modules activity. The density scatter plot comparing edge weight before and after treatment. PCC: Pearson's correlation coefficient of the scatter plot; Rsq: r-square measure of fitness of linear regression fit with a straight line.

FIG. 39 shows representative raster plots of individual spheroids and Modular Neuronal Network (MoNNet). Left to right: Neuronal spiking raster plots extracted from spheroid and MoNNet. Images shown are maximum projection across time and corresponding intermediate peak signal-to-noise ratio images from the CalmAn based analysis pipeline for activity source extraction. The neurons belonging to same spheroids are grouped together in raster plots.

FIGS. 40A-40B show three distinct phases of MoNNets. (FIG. 40A) Neuronal activity traces extracted from MoNNets in three phases of network activity. Neurons belonging to same spheroids are grouped together in same color. (FIG. 40B) Left-to-right: Histograms comparing the pooled data in three phases: local(green) and global(blue) average PW correlation; local(green) and global(blue) network efficiency of weighted functional graphs; number of detected modules; modularity measure Q. Statistical significance is defined as: *p<0.05, **p<0.01, ***p<0.001, ****p<0.0001. Sonsignificant differences are not marked. Note that the global activity correlation from FIG. 34D is plotted again for completeness.

FIGS. 41A-41C show characterization of MoNNet activity patterns before and after treatments with synaptic and ion channels inhibitors. (FIG. 41A) Control experiment. From left to right: Representative examples for raster plots and correlation graphs in Phases I and II. Before and after treatment comparison of average pairwise correlation, network efficiency, firing rate per minute and full width at 75% of the maximum dF/F peak. (FIG. 41B) Representative examples of raster plots and correlation graphs for every drug condition. (FIG. 41C) Summary table with all average data, standard deviation, p-values and number of experiments (N), for every condition and analysis.

FIGS. 42A-42C show immunolabeling of spheroid sections and electrophysiological recordings of spontaneous action potentials. (FIG. 42A) Immunolabelling of PH3 and Caspase demonstrates existence of proliferating cells, and general absence of cell deaths. (FIG. 42B) Sparsely labeled MoNNet preparation derived from Thy1-eYFP transgenic mice. Scale bares are 100 μm. (FIG. 42C) Top, left-to-right: Representative trace of a cell-attached recording from MoNNet, showing spontaneous action potential (AP) at ˜1.5 Hz; representative traces showing robust AP responses to 400 pA depolarizing current steps (reference lines: −70 mV and 0 mV levels); representative traces of voltage-step (−100 mV to 50 mV, cells held at −70 mV between voltage steps) evoked currents showing robust voltage-gated sodium currents and voltage-gated potassium channel currents. Bottom, left-to-right: representative 30 s trace of a neuron held at −70 mV showing high frequency, small amplitude excitatory synaptic responses; representative 30 s trace of a neuron held at 0 mV showing very large, high frequency (>3 Hz) spontaneous inhibitory synaptic events; representative 100 s trace of a neuron held at 0 mV showing large, high frequency spontaneous inhibitory synaptic events and its reduction in presence of CNQX (10 μM).

FIG. 43 shows average pairwise correlation of MoNNets derived from WT CD-1 vs. WT C57BL/6J. Local and global average pairwise correlation of MoNNets derived from WT CD-1(red) and C57BL/6J(blue).

### DETAILED DESCRIPTION

Those skilled in the art will understand that this invention may be embodied in many different forms and should not be construed as limited to the embodiments set forth in this application. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description.

The drug development for psychological and neurological disorders has essentially stalled due to high rate of failures in clinical trial. Our NNet approach, which can be derived from patient specific iPSC cells, provides an effective mean for modeling of brain functional complexity and disorders, while providing systems-level observation and manipulation capabilities. NNet, in combination with microfluidics and miniaturized imaging approaches, can yield a compact and effective screening platform for psychoactive compounds. In addition, we expect NNet to also inspire next generation of machine learning approaches by discovering principles of higher order brain function.

Compared to brain organoids, the disclosed NNet subject matter may provide the following advantages:

Time of production: To grow mature functionally-active organoids takes around 4-6 months while NNet will take only a matter of days or weeks.

Accessibility: Brain organoids are opaque and dense, making very difficult to study the internal connectome organization. The NNets exposes all the connections for easy observation and manipulation.

Plasticity: Formation of the functional neuronal networks in Brain organoids are not easy to control, whereas NNet are amenable to rational designs by employing different shape and patterns of molds and/or the guided axonal connectivities (e.g., by using Netrin).

Heterogenity: So far, only Bagle et al 2017 (Nat Methods. 2017 July; 14(7):743-751. doi: 10.1038/nmeth.4304. Epub 2017 May 10.) have been able to grow hybrids organoids with two different regions, but the NNet disclosed herein permits combine in even more complex and controlled way using neurospheres derived from different brain regions.

Advantages of the NNet approach may include: (1) arbitrary control of the size and patterns of NNet, and therefore their complexity and correspondence to higher order brain functions; (2) precise manipulation (using optical means) and observation (using large field-of-view imaging) access to individual neurons, connections, modules and even the entire network, and (3) multi-scale intra-/inter-/supra-modular activity and connectivity patterns to better model brains; (4) amenability to automated high-throughput screening platforms (e.g., using microfluidics) for psychoactive compounds; and (5) access to patient-specific analysis.

Such features advance the ability to model brain complexities and its disorders. Being open preparation, NNet are easy to integrate in microfluidics based platform for systematic screening of psychoactive compounds for brain diseases. Patient specific NNet can be sourced from iPSC-derived neuronal progenitors to implement personalized medicine assays. We have also discovered precise sample-invariant quantitative descriptors that correspond to higher-order in vivo brain functions including formation and maintenance of neuronal ensembles, memory storage and recall.

Applications include: (1) modeling of complex brain disorders to understand the pathophysiology of neural circuits at systems level, (2) automated and high-throughput drug screening, (3) personalized medicine for psychiatric disorders, and (4) improving machine learning approaches by combining with artificial neural network theories.

In general, pharma industry will be most interested in using these methods for better screening of potential drugs before clinical trials. In addition, companies aiming for personalized treatment may benefit from quick assays to assess the efficacy of various available options, and this will provide models for basic research on the complex physiology of brain architecture.

### Self-Organized NNet

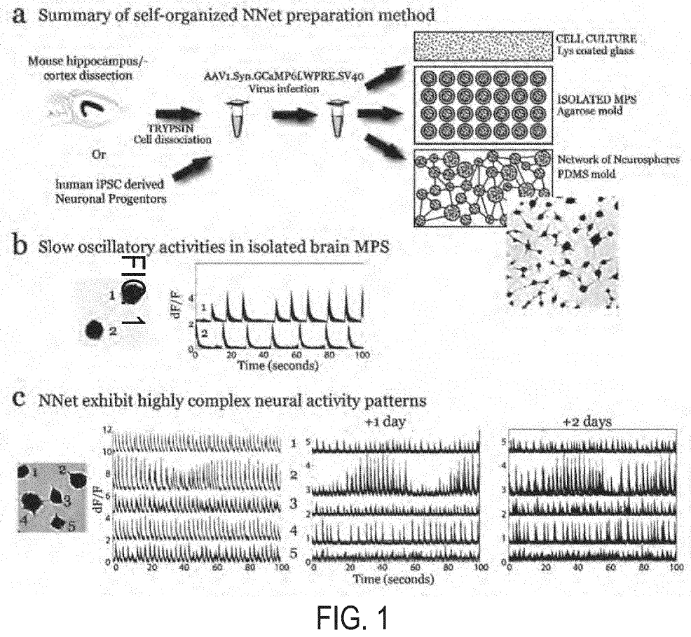

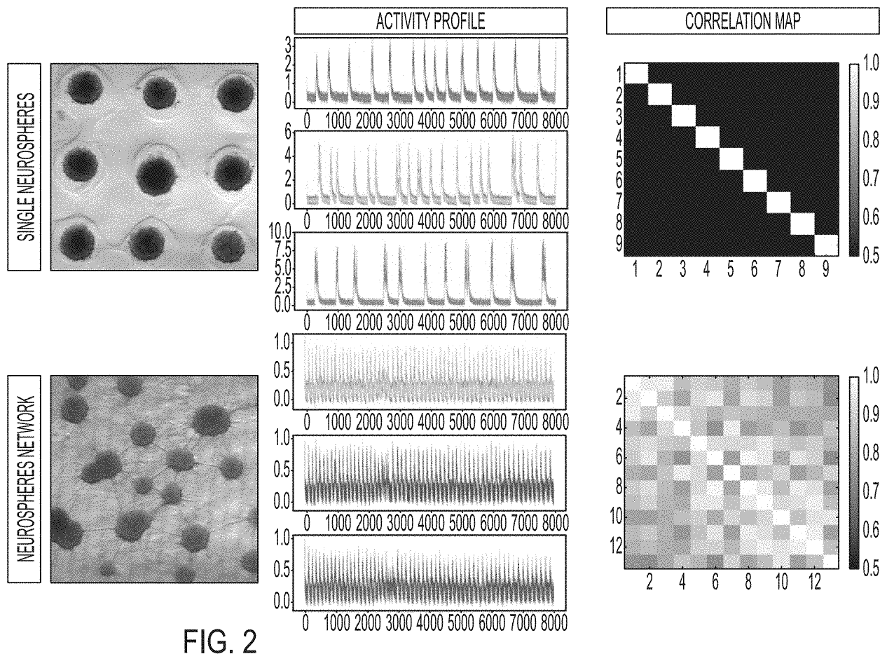

In its simplest form, NNet can be developed by plating of neural cells on PDMS molds of specific pattern and size. The starting cells can express calcium indicator GCaMPs (using standard AAV vectors or transgenically) as proxy for neuronal activity. Calcium sensitive dyes can be used as alternative. The activity can also be recorded by an array of electrodes. We implemented the NNet and found more complex activity patterns (FIG. 1 and FIG. 2). Due to open-access layout, NNet is amenable to cell-type specific activation/deactivation (e.g., using optogenetic, or light-sensitive caged compounds as described in the accompanying IR) for model brain function and disorders (e.g., Autism Spectrum Disorder is caused by imbalance in Excitation/Inhibition).

### Supervised NNet Assembly

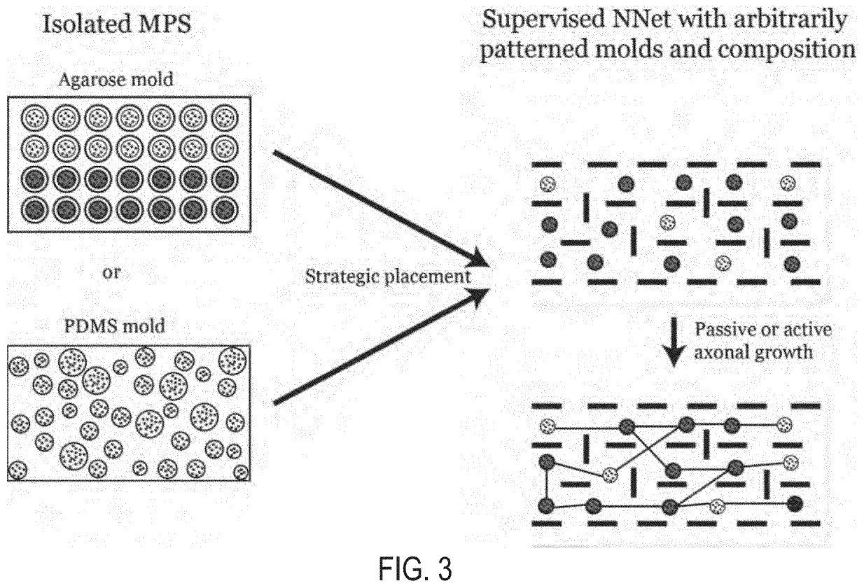

In this approach, one can design any specific NNet configuration. NNet forms in two steps: cells aggregate to form individual 3D neurosphere/unit, followed by their inter-connection. This allows us to collect individual neurospheres and re-position them strategically on a patterned PDMS mold for inter-connections passively or actively by using guidance cues like Netrins (e.g. using soaked microbeads) (see FIG. 3). We found that neurospheres collected at least before 12 hours (after plating) establish inter-connections.

### Heterogeneous NNet

Supervised NNet allows assembling of heterogeneous NNet by combining individual units from different sources (FIG. 3 in attachment), such as normal and disease models, or different brain regions. Such preparations allow effective investigation of brain disorder mechanisms. For example, most brain disorders start in small regions, followed by their spread across the entire brain. This phenomenon can be easily capture by generating heterogeneous NNet composed of only a few nodes from diseased sources.

### Quantitative Descriptors of Higher Order Brain Functions

One major problem in the MPS field is how to extract meaningful quantitative descriptors that correspond to higher-order brain functions. We have identified several such features, including: (1) Formation of neuronal ensembles (i.e. correlated cells) which are the unit of computation and information storage. (2) Maintenance of ensembles over time as a measure of stability of information encoding and processing. (3) Pattern completion, by activating part of an ensemble to capture model memory recall. (4) Integration of function and the underlying structure to model causal structure-function relationships.

### Applications

The NNet approach may be used for modelling of brain and the disease pathophysiology, personalized medicine by patient-specific NNet for screening of psychoactive drugs, and next generation artificial neural networks.

For drug screening, brain cells may be obtained (or derived from stem cells or iPSC cells) of mammals presenting a biomarker indicative of a brain disorder, where the biomarker may be a gene mutation or a biologically active protein, or any other physiological material such as polynucleotides that indicate a disease state. For example, the biomarker may be a neurotransmitter-related protein or molecule (for example parvalbumin, somatostatin, glutamate, GABA, or dopamine) or a transcription factor or an effector gene marker.

The compound being screened may be a small molecule, a polynucleotide, a protein, or a virus particle. For example, the compound may be a DNA based or RNA based active compound, or a biologically active protein such as an antibody.

The brain disorder against which the compound is screened may be, e.g., schizophrenia, epilepsy, autism, Parkinson's disease, depression, or a neurodegenerative brain disorder such as Alzheimer's disease.

The screening method captures signals (e.g., low average pairwise correlation of activities) characteristic of the brain disorder. The signal may be one or more factors, preferably the patterns of activity of individual brain cells and their evolution over time, synchronization or correlation of activity of all brain cells or brain cells belonging to individual units over time, formation and stability of neuronal ensembles, stable temoral activity sequences, or the population level activity evolution over time, as quantified by dimensionality reduction. The dimensionality reduction may be, e.g., principal component analysis, independent component analysis, or tSNE, or the relationship of activity patterns to underlying molecular identity of brain cells.

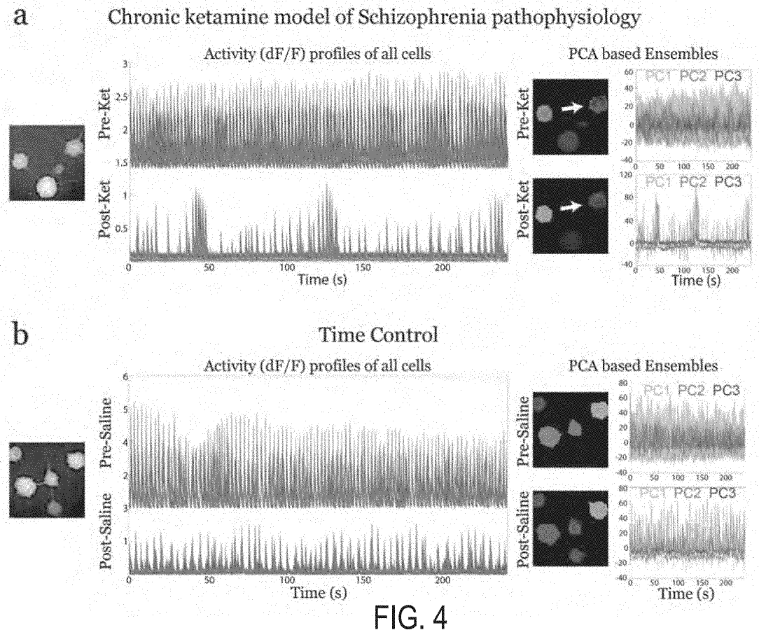

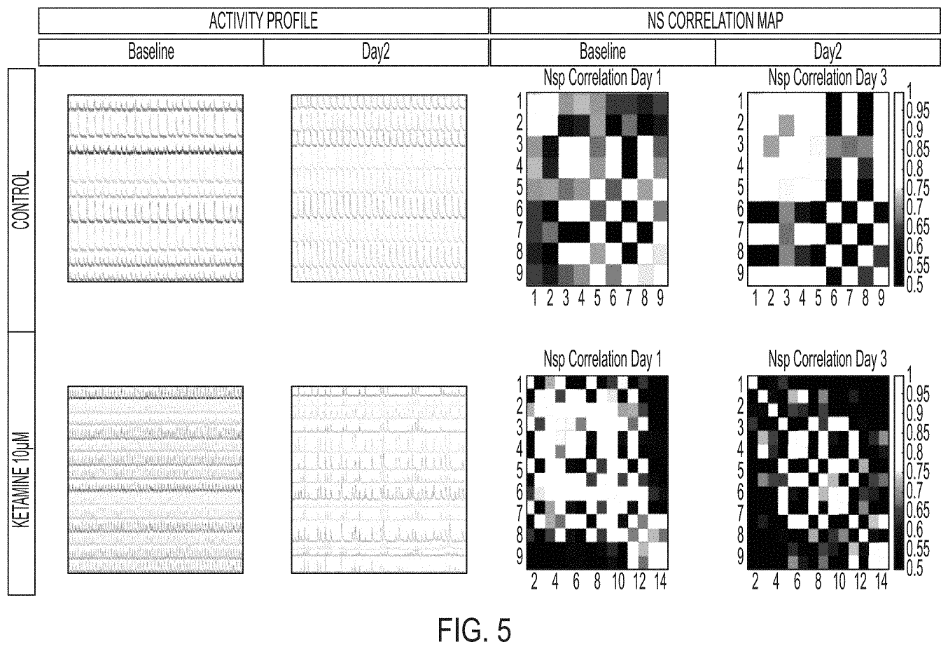

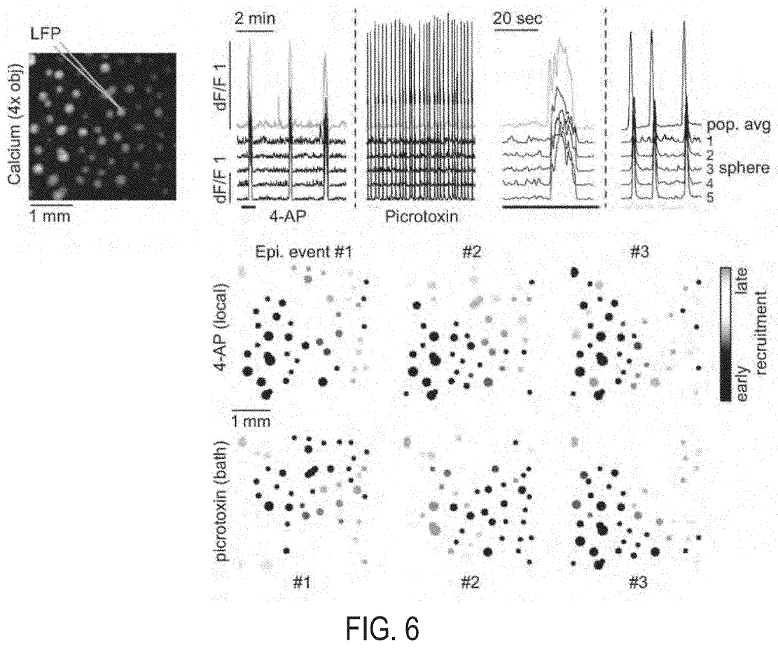

As explained above, NNet approach is validated for effective modeling of Schizophrenia pathophysiology (see FIGS. 4 and 5) and epilepsy (see FIG. 6).

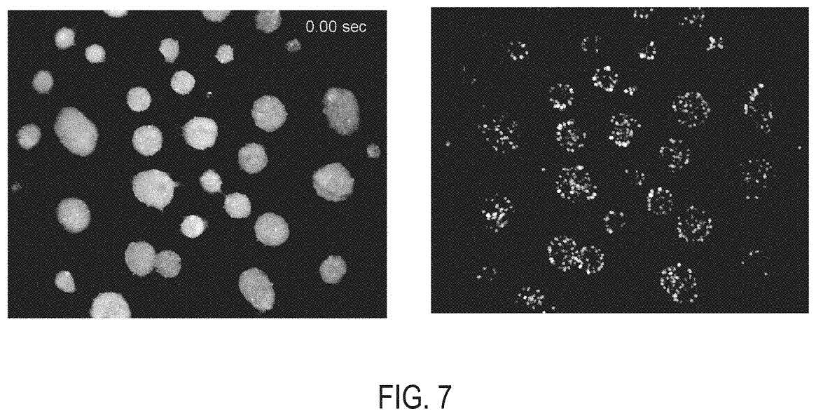

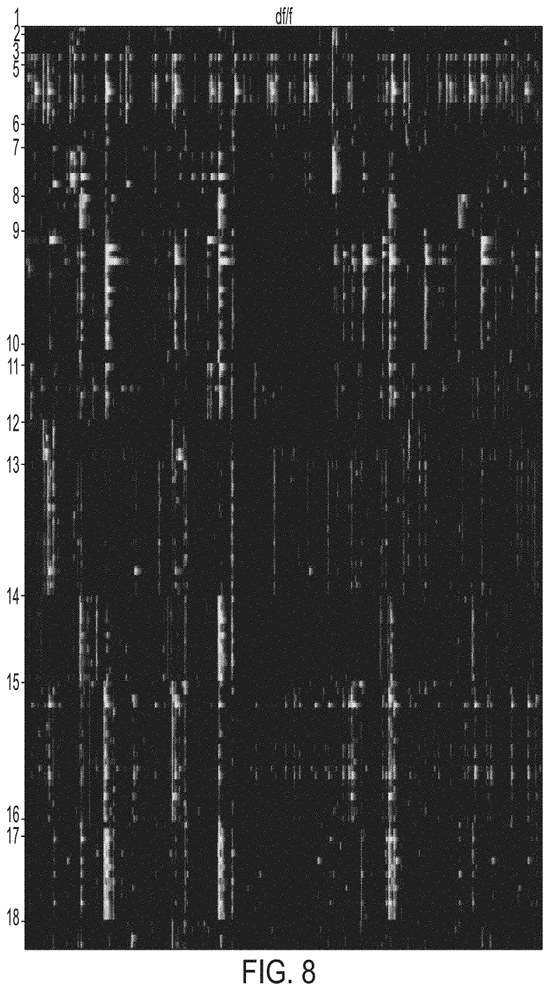

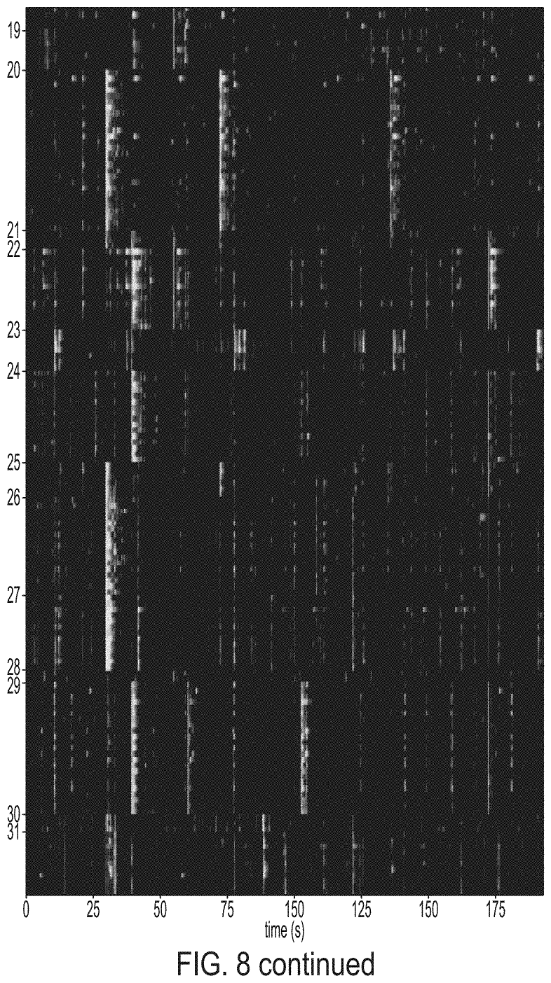

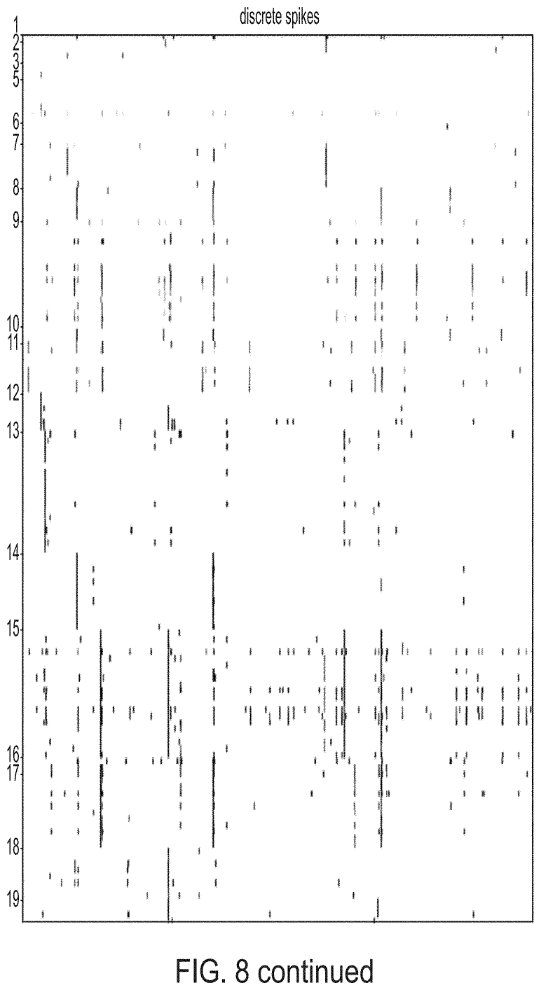

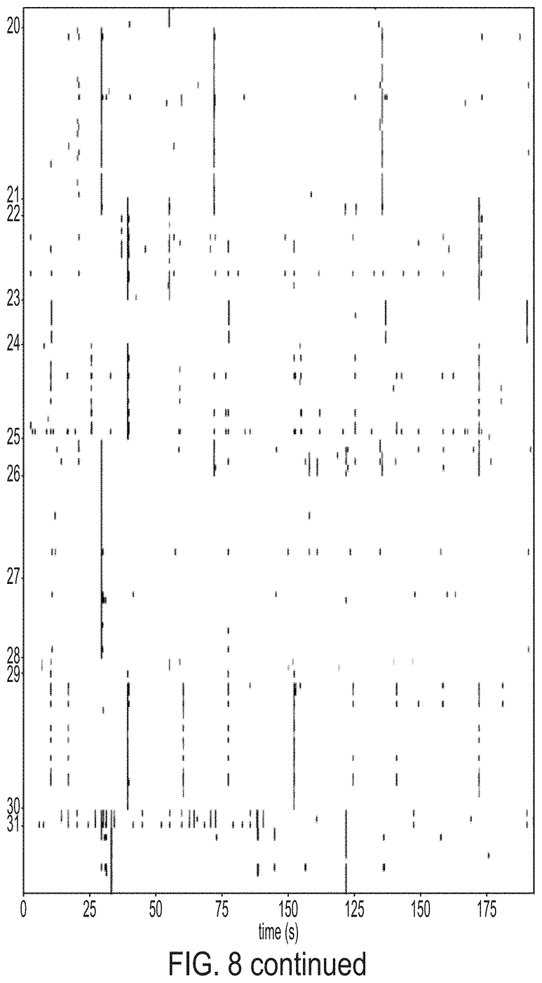

The imaging data shown in FIG. 7 resulted from Calcium Imaging data Analysis (using CalmAn based-pipeline). FIG. 8 shows activity traces of cells identified in FIG. 7. In FIG. 8 each row represents a brain cell. Shown on the left of FIG. 8 is a heatmap showing level of activity (i.e. delta F over F, which is a standard way to quantify change in signal). Shown on the right of FIG. 8 is discretization of these signals, i.e., discrete spikes, to identify when a brain cell fires (or becomes active).

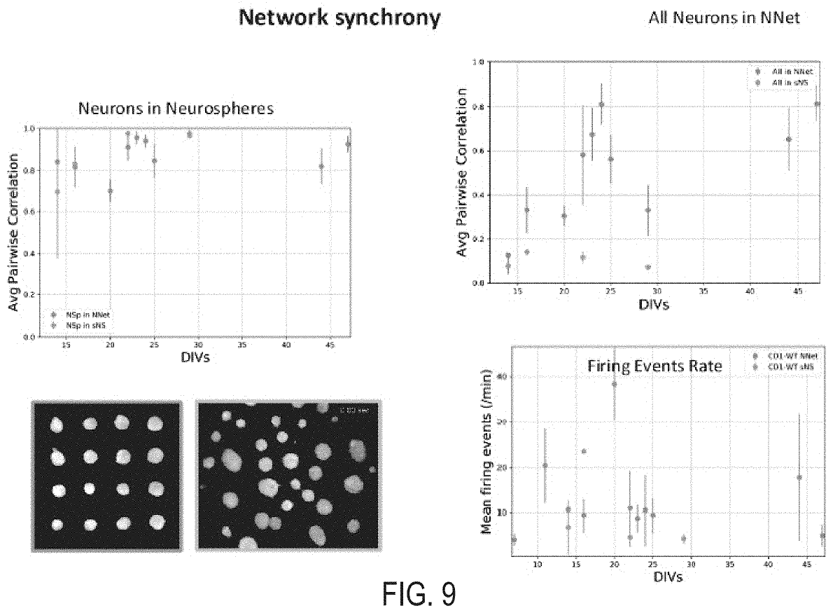

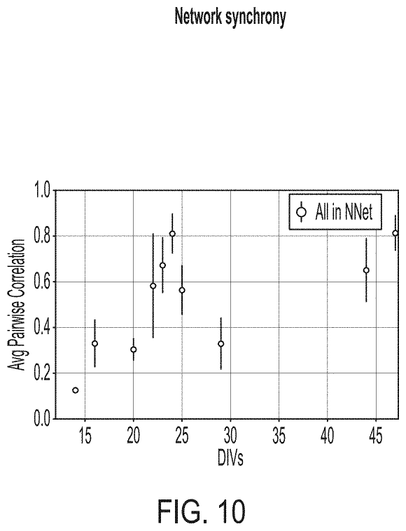

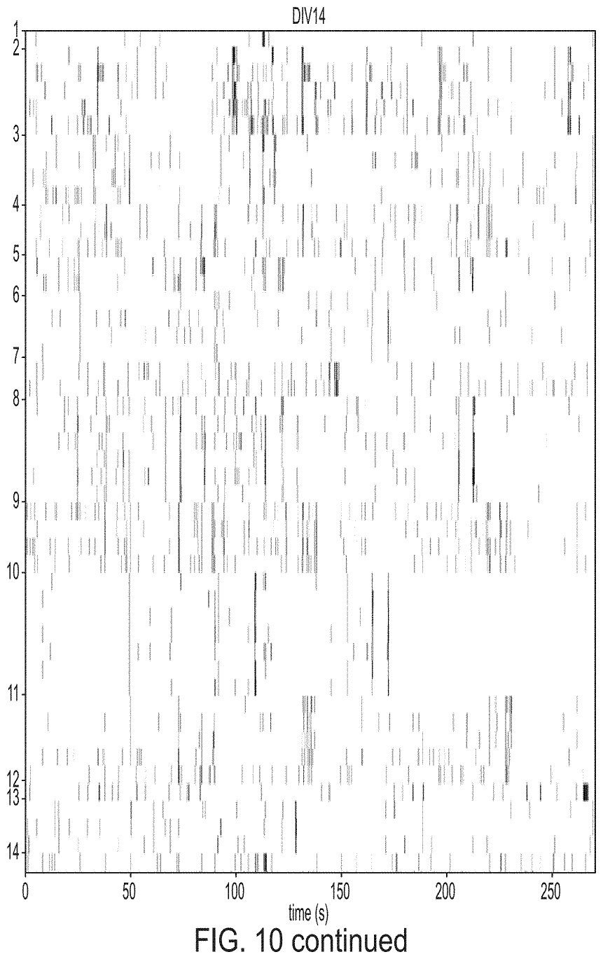

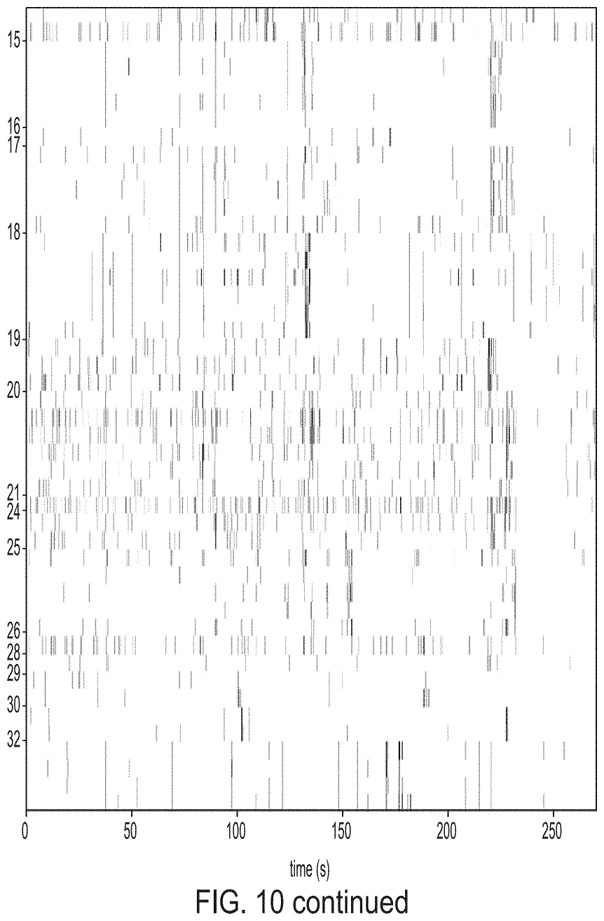

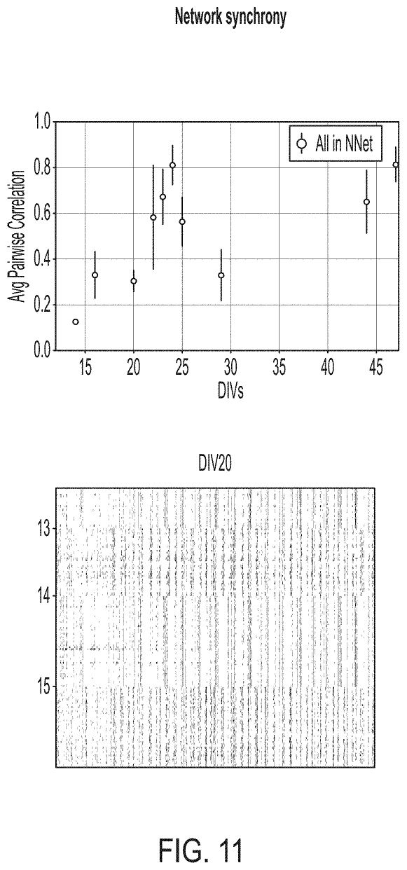

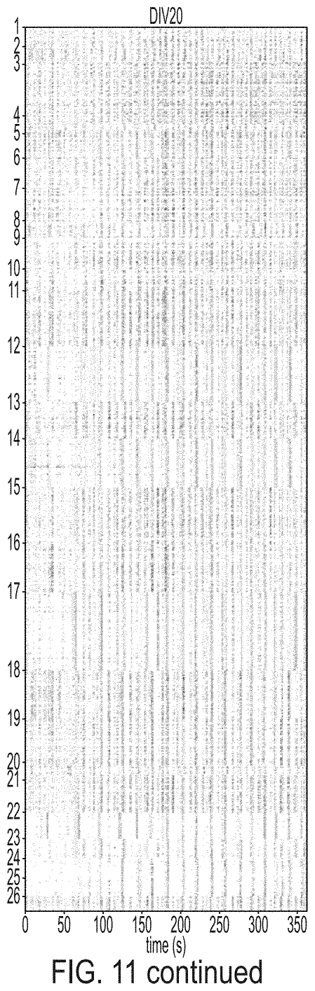

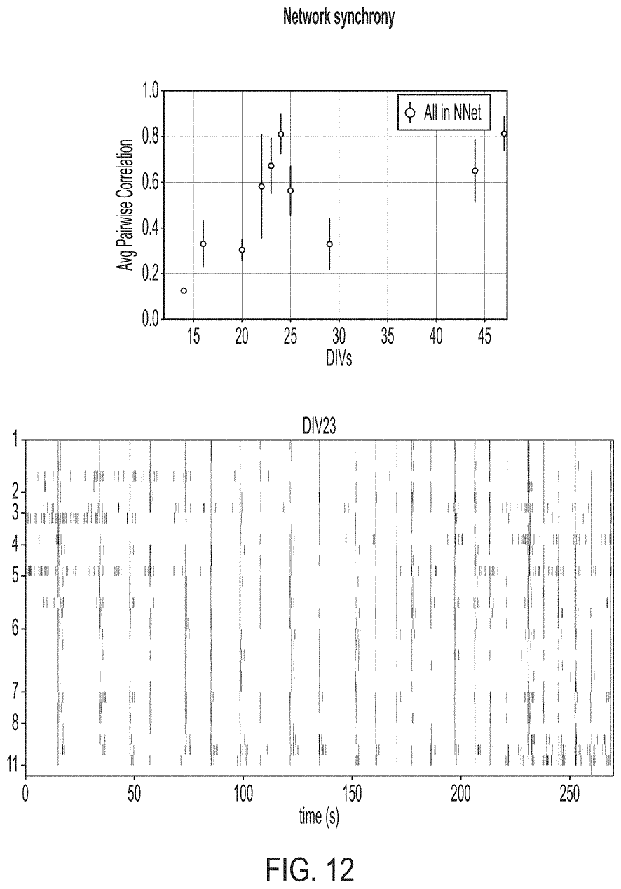

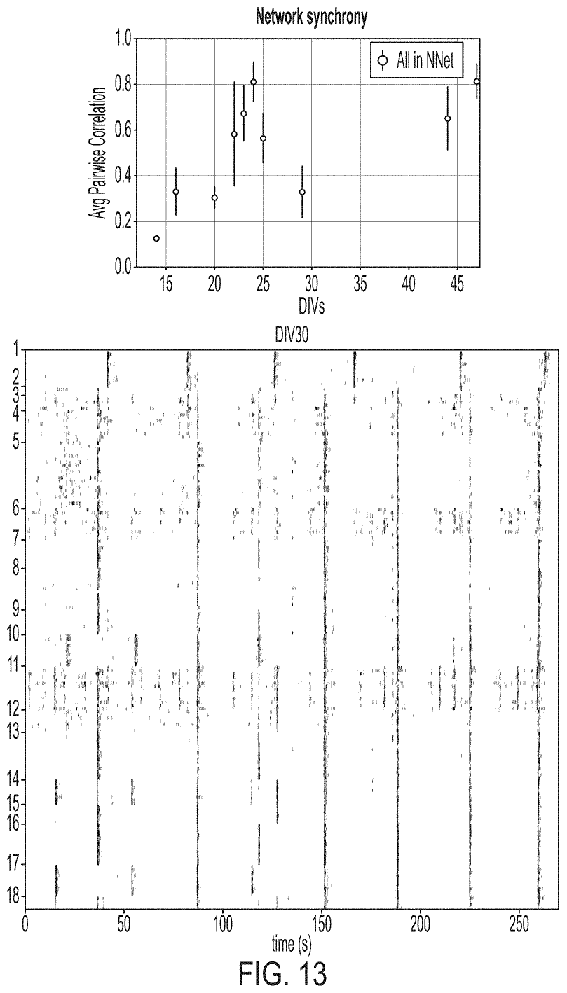

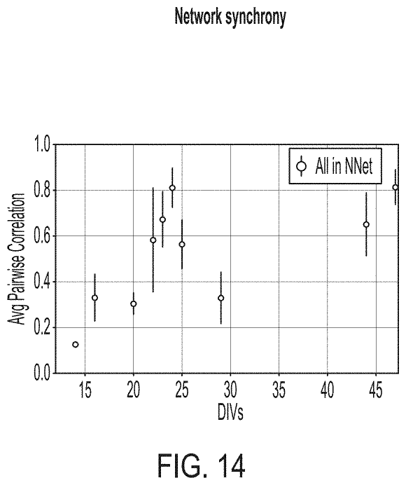

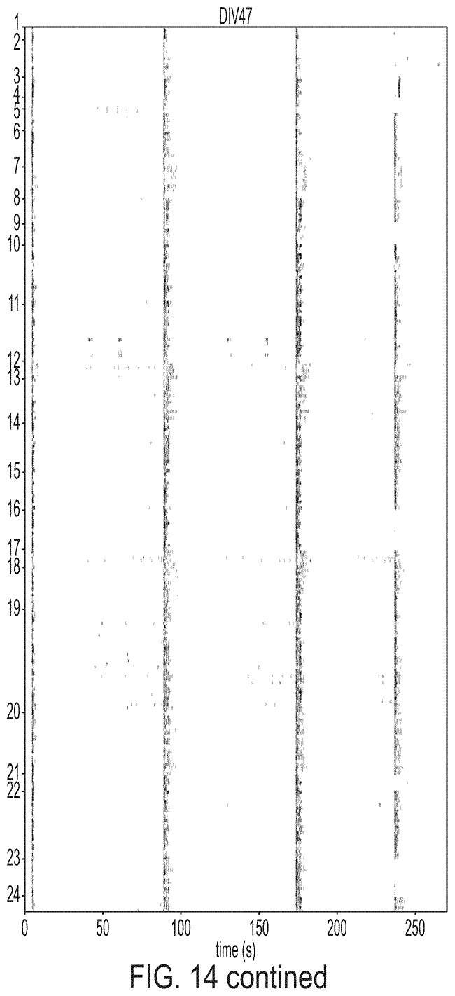

FIG. 9 plots the synchronization of activities of brain cells (quantified as average pairwise correlation) and compares two situations: orange when the individual units are not connected to each other, and cyan when individual units are connected to each other. The plot on the left (titled Neurons in Neurosphere) plots average correlation among only those neurons that belong to the same unit. The plot on the right (titled All Neurons in NNet) is for all neurons without any restriction. The bottom-right graph plots average number of firing events. In the plots “DIVs” means days in vitro i.e. days after culturing. It is notable that in left plot the correlation becomes high very quickly and remain high, whereas in right the correlation increases slowly, and then decreases. Orange is meant to show that this effect is not seen for preparations that are not connected. Activity patterns at different days after culturing (DIVs) are shown in FIG. 10 to FIG. 14. It is notable that even though in higher DIVs the correlation is low, as in the beginning DIVs, the activity is much more structured, indicating development of distinct patterns and connections over time, mimicking developing brain circuits.

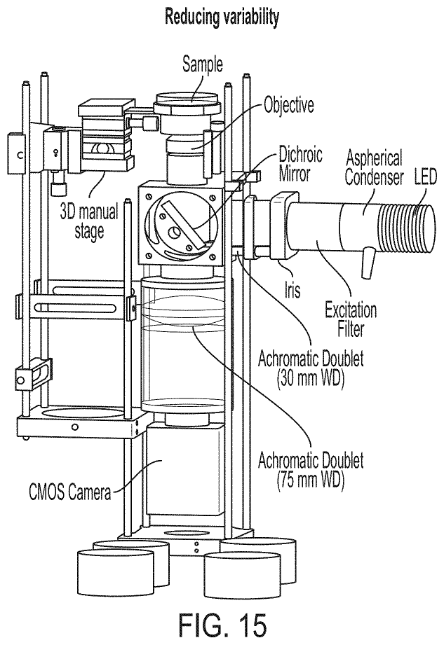

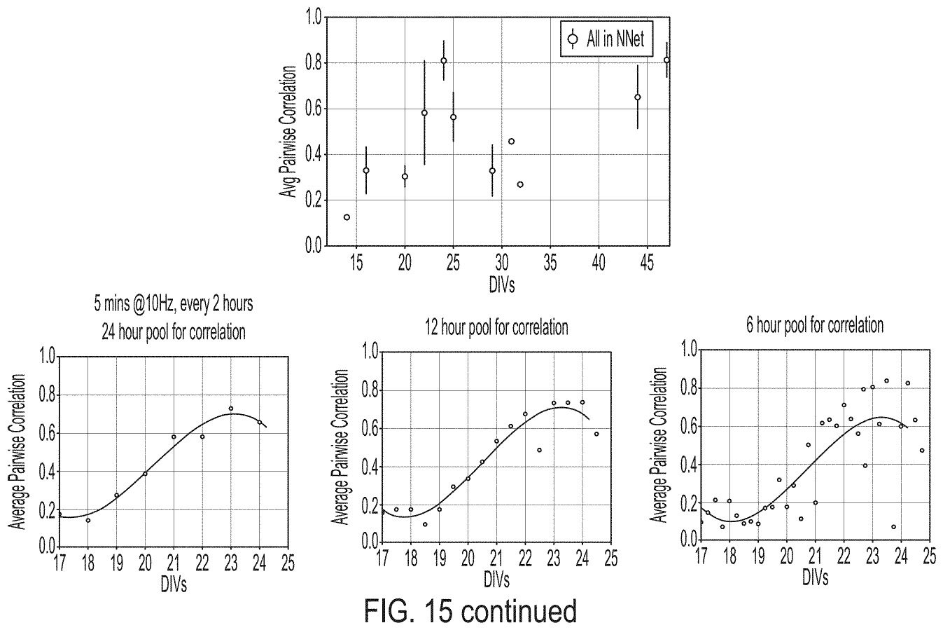

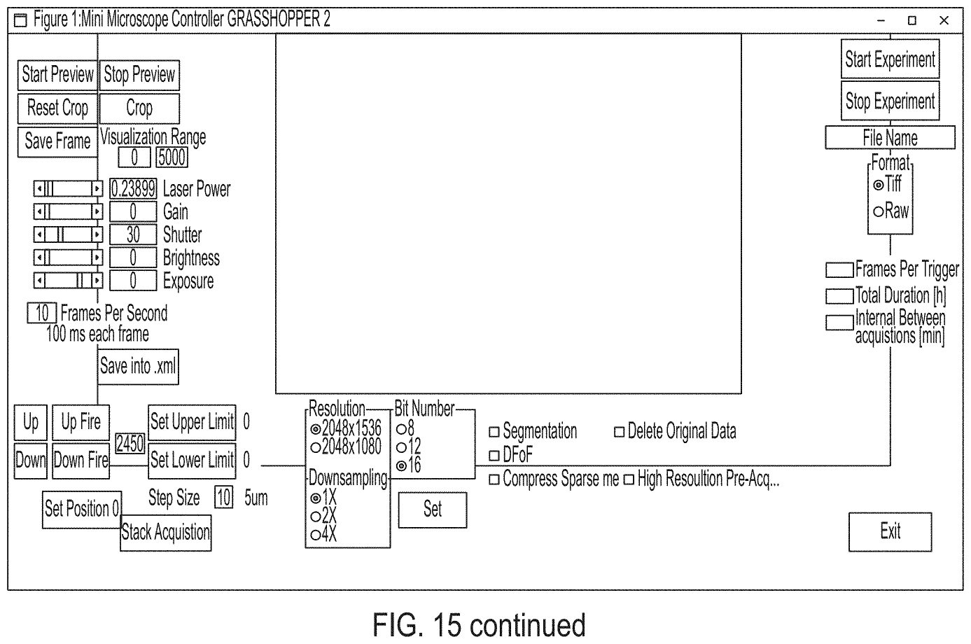

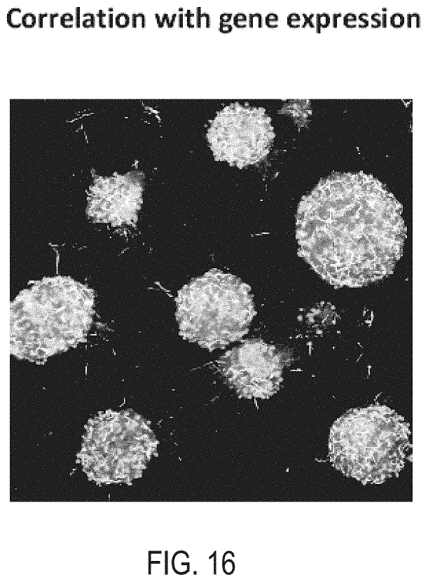

FIG. 15 shows the results obtained when the experiment was done using a custom-made miniaturized microscope placed directly in an incubator. The live recording was conducted for about 5 mins at high speed at defined intervals (e.g. every 2 hours). The plots on the bottom show one example of pairwise average correlation of one network over time. As noted in FIG. 15, the sample was recorded for about 5 minutes, every 2 hours. Different plots are calculations of average correlation by pooling in time points.

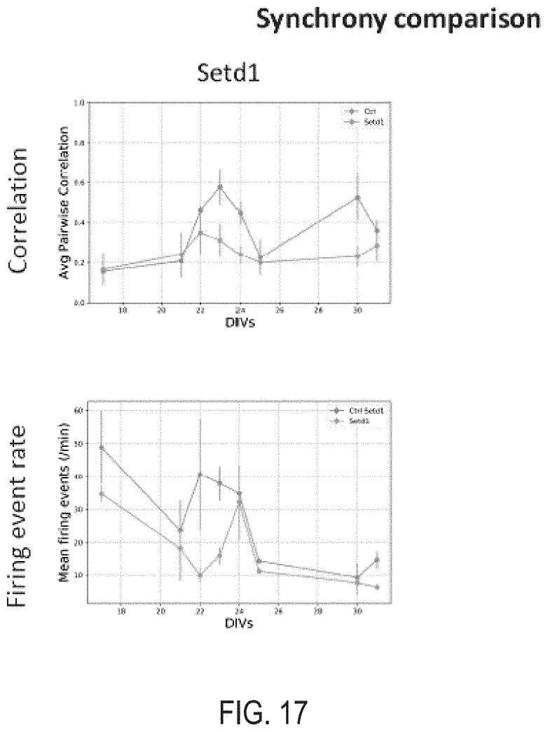

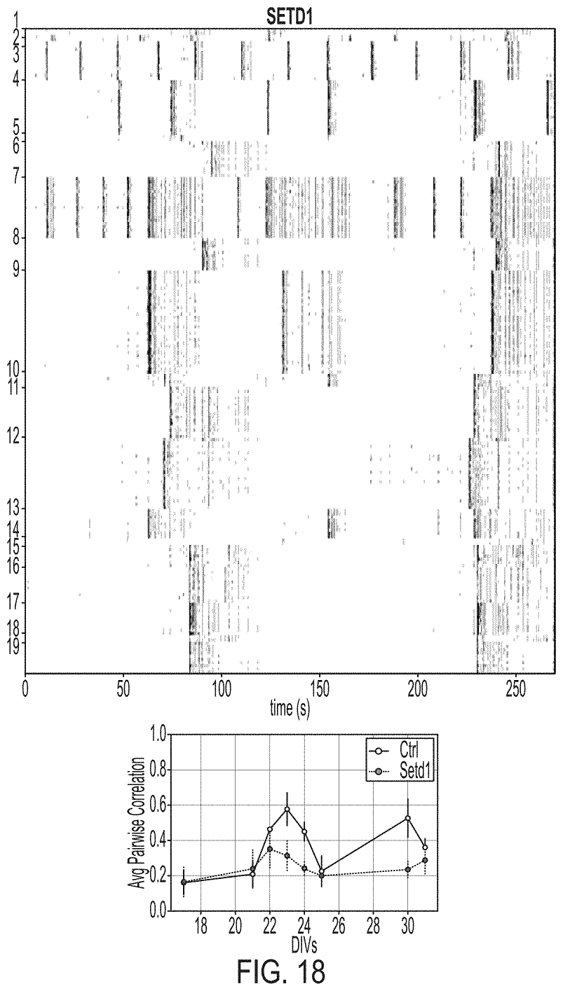

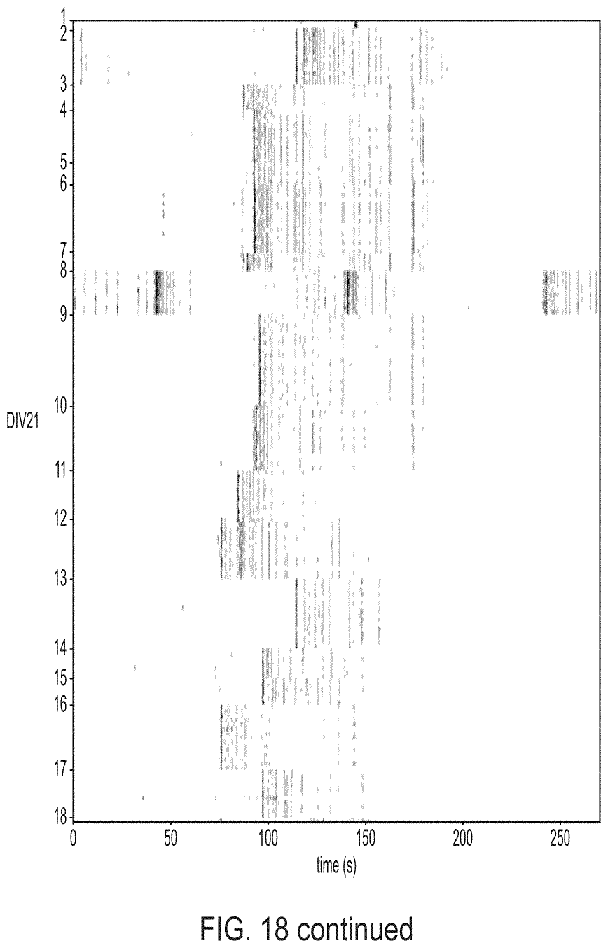

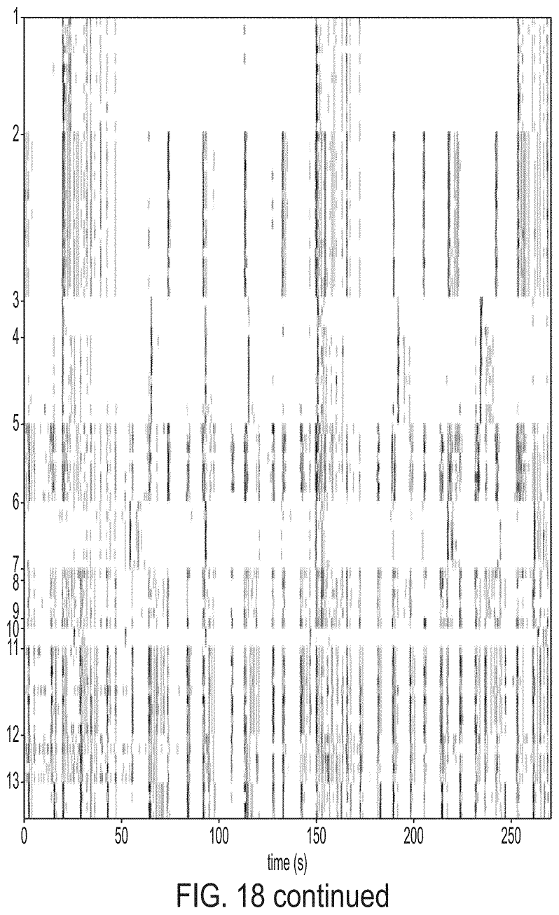

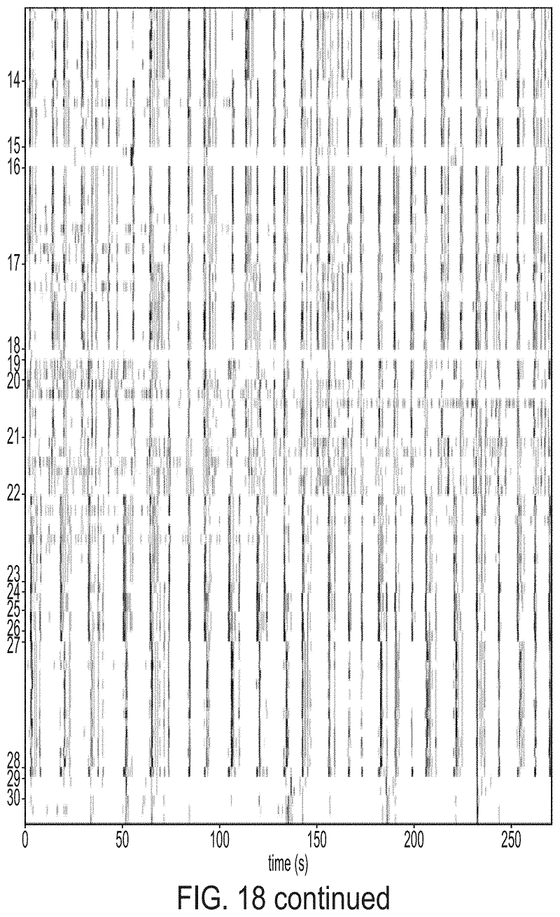

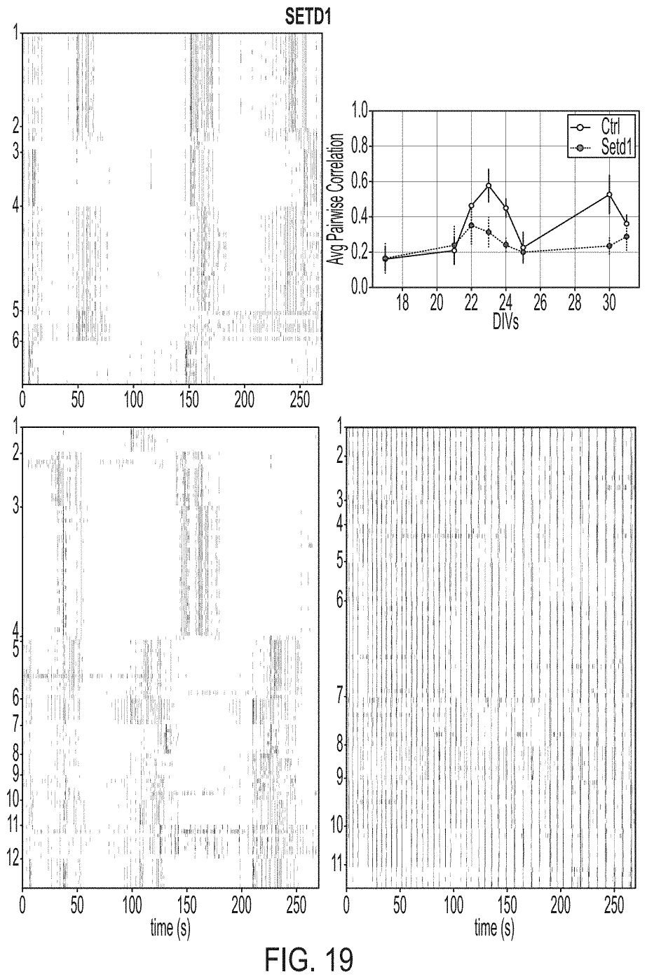

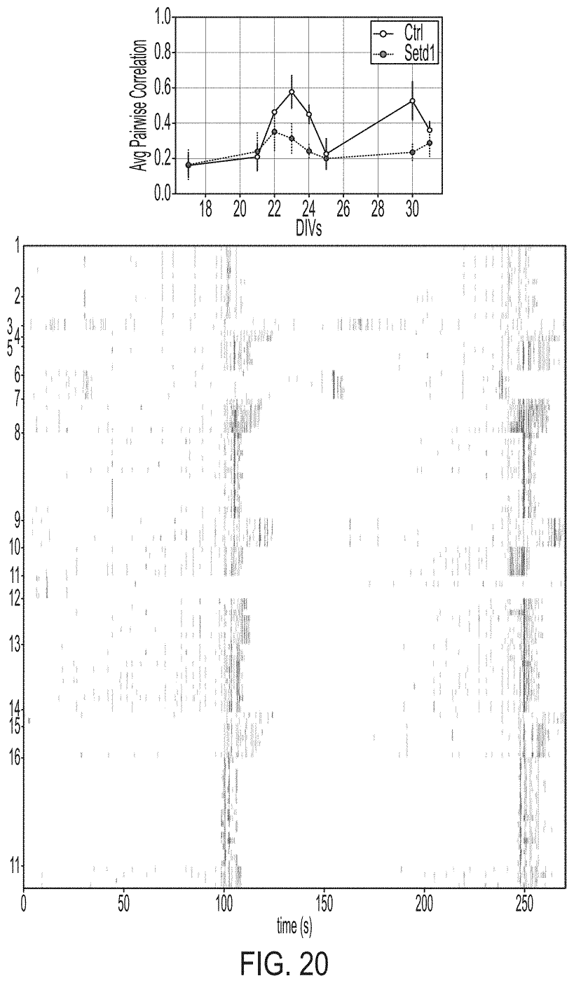

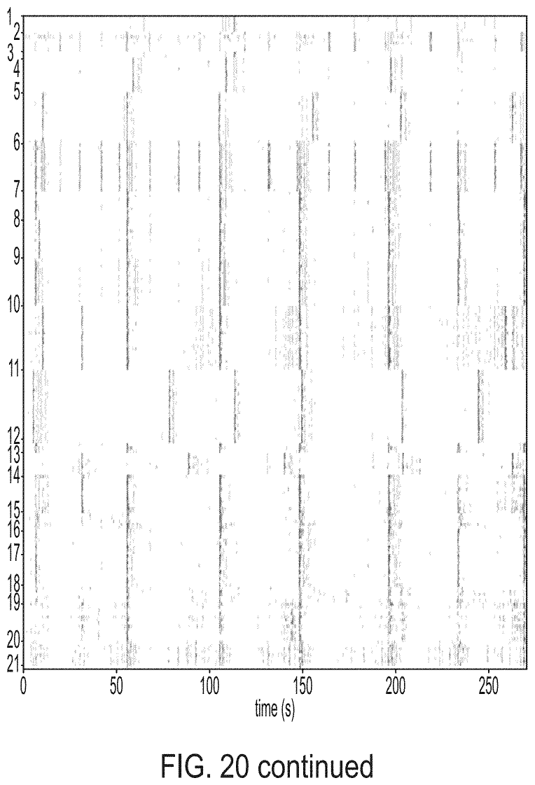

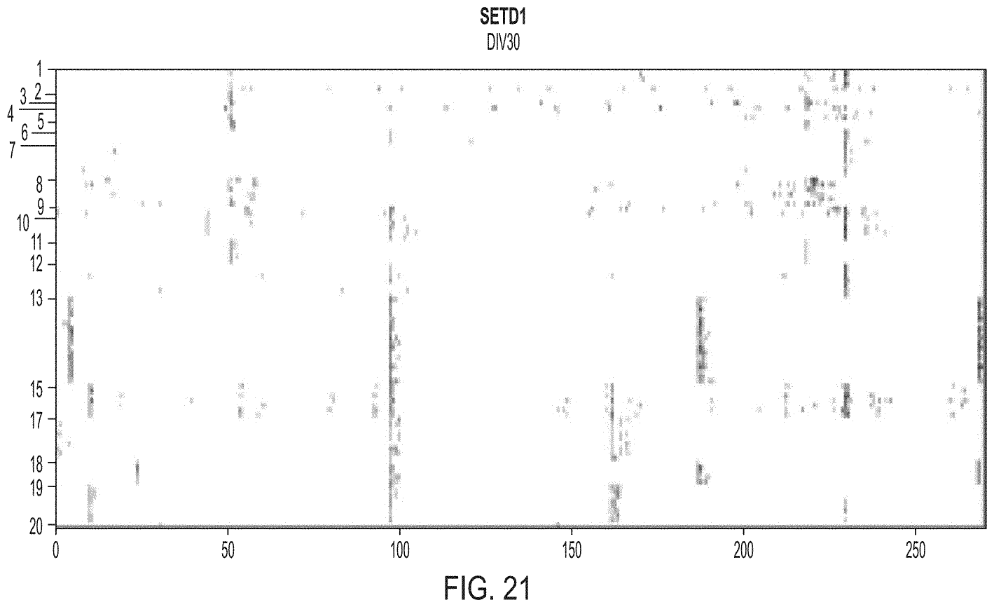

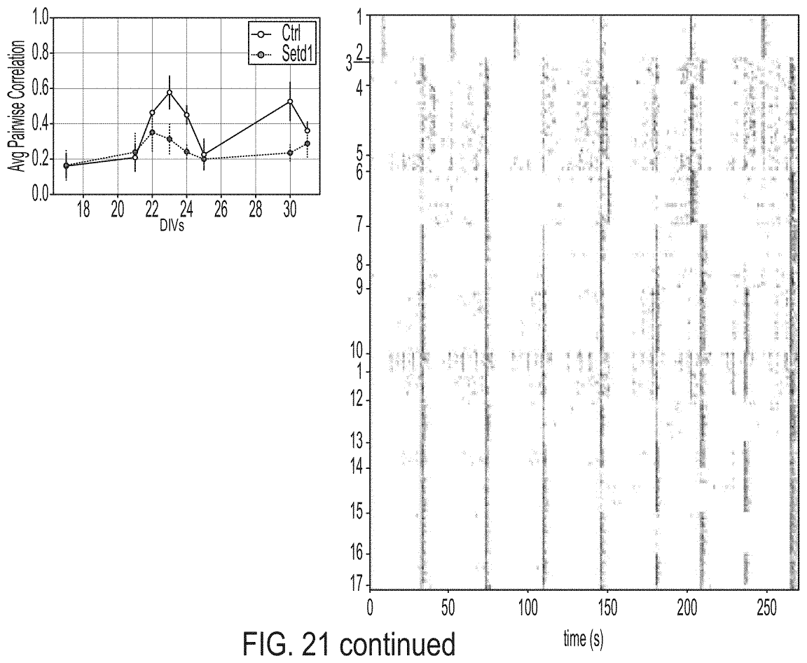

FIG. 17 compares synchrony of a control and a SETD1 mutant (a model of Schizophrenia). The top graph shows that the diseased ones do not develop high synchrony. FIG. 18 to FIG. 21 clearly showing striking differences in structure of activity patterns for diseased (on the left) and control (on the right, after animation). These differences, i.e., in synchrony and also patterns of activity, can be used to assess if treatment by a specific drug candidate can restore them.

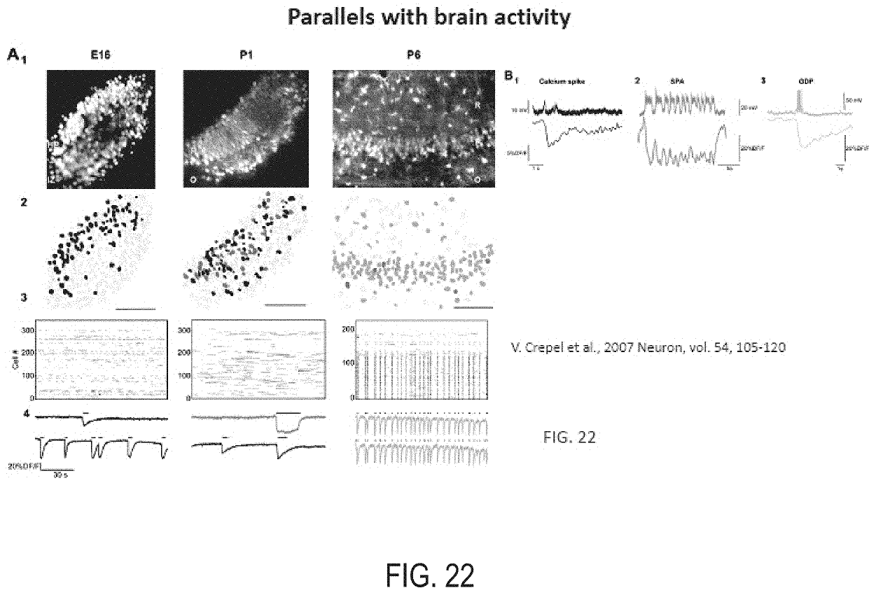

FIG. 22, FIG. 23, and FIG. 24 show that the NNet preparations capture the kinds of patterns that are known from real brains in literature (shown). This demonstrates that one can transfer the knowledge gained from these cultures to brain defects.

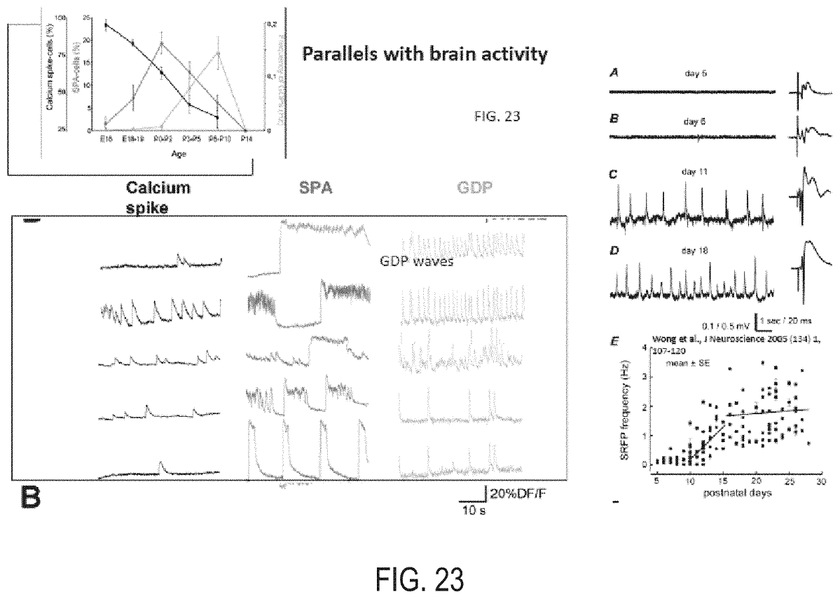

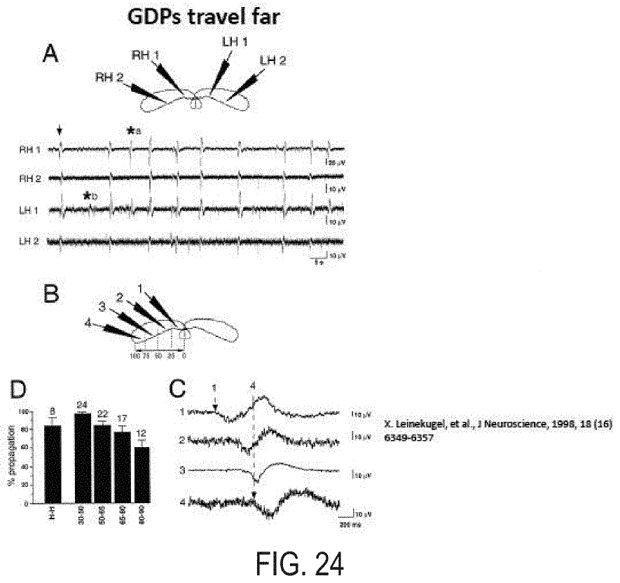

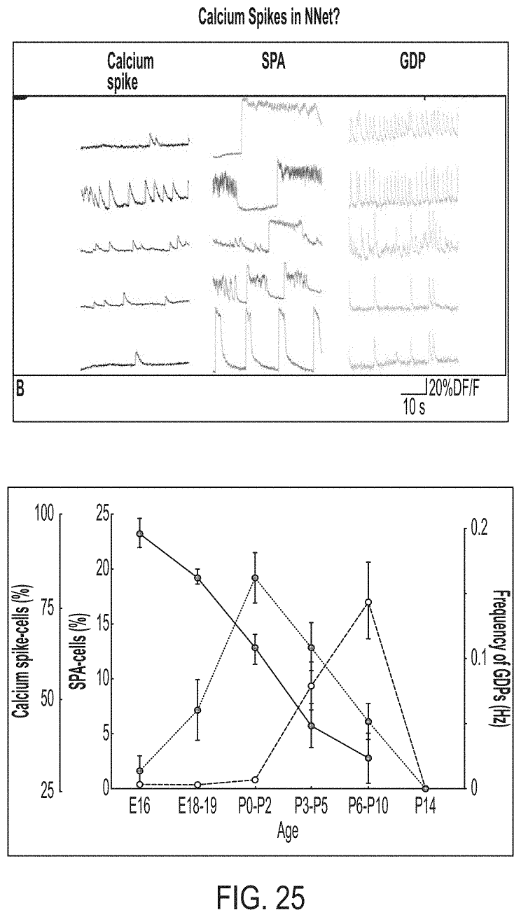

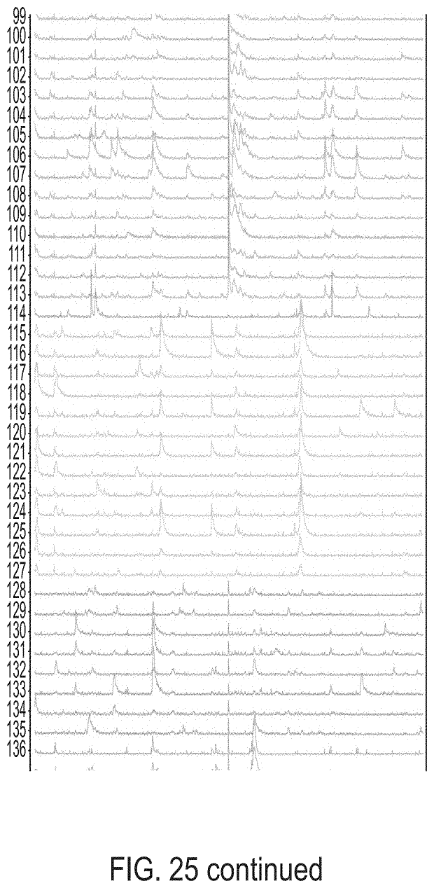

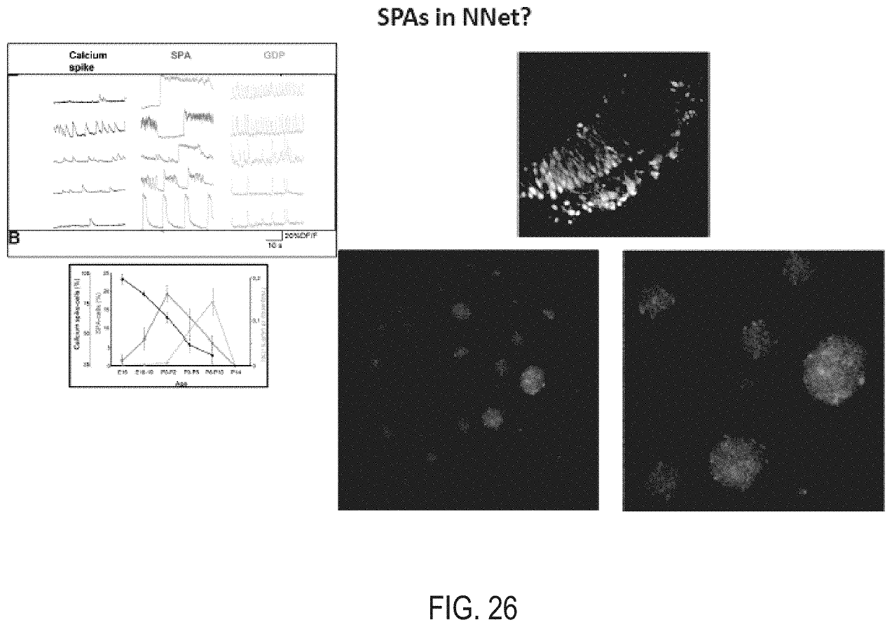

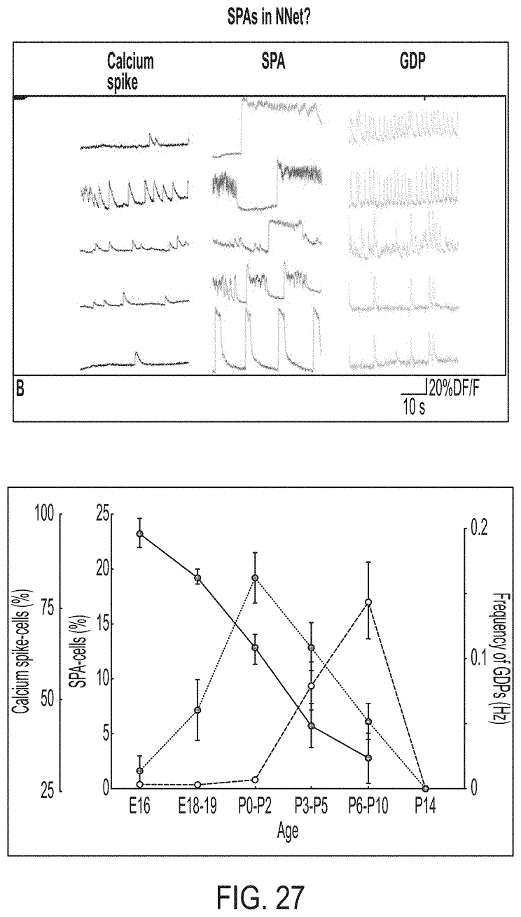

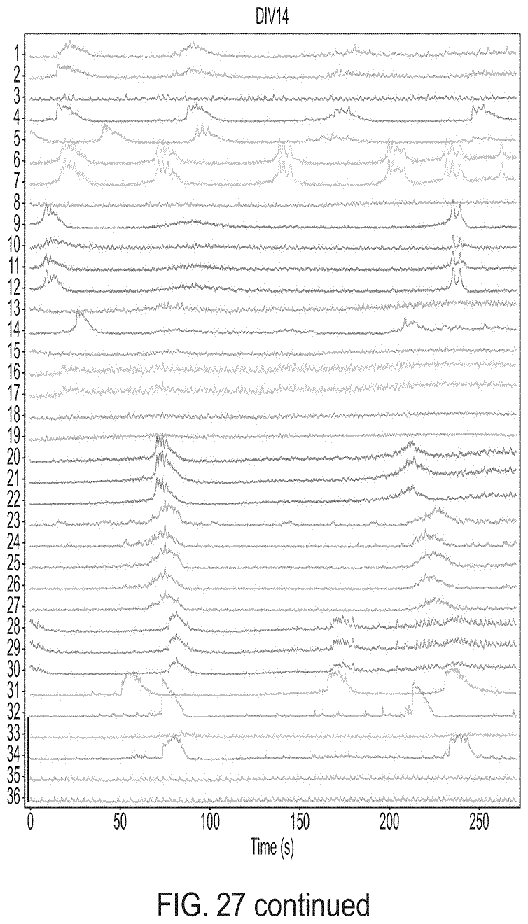

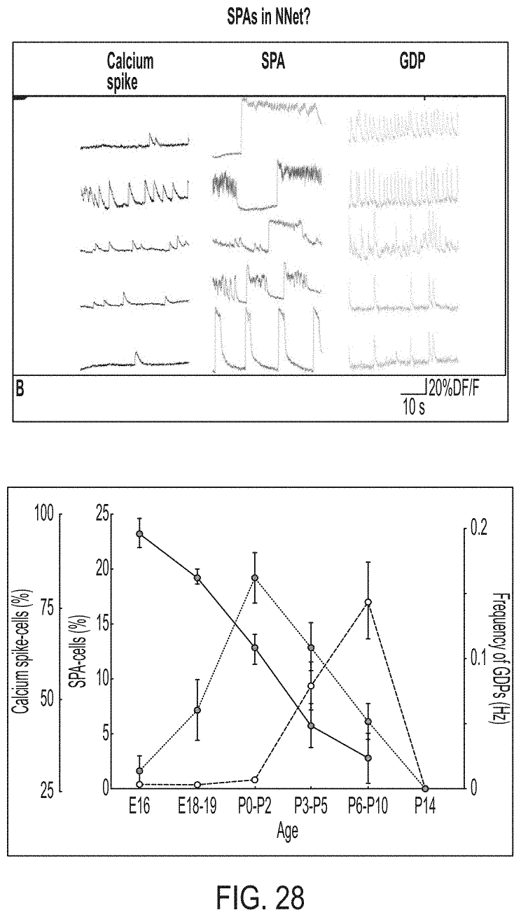

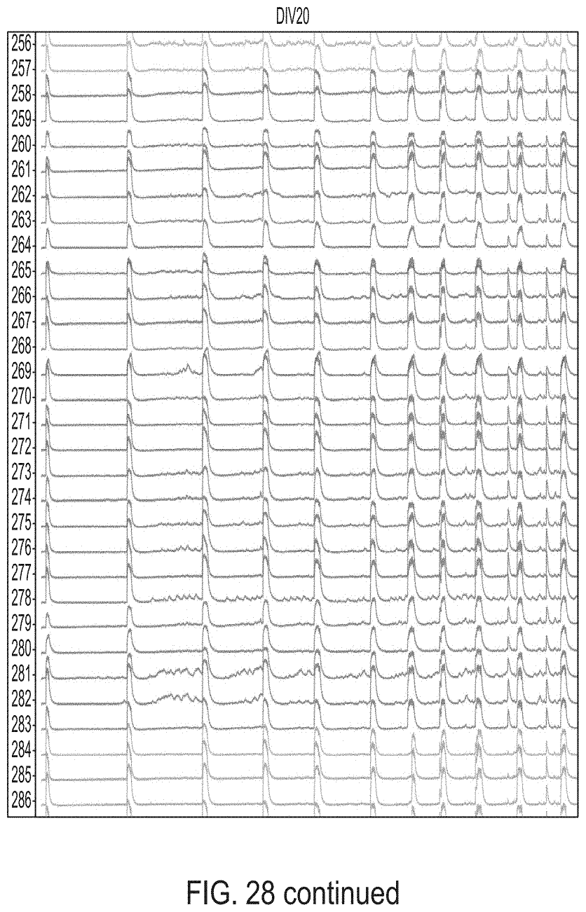

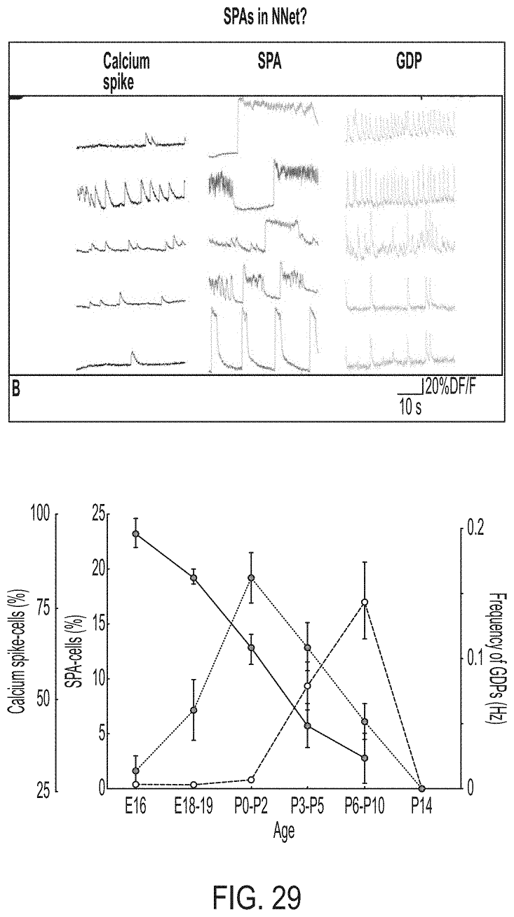

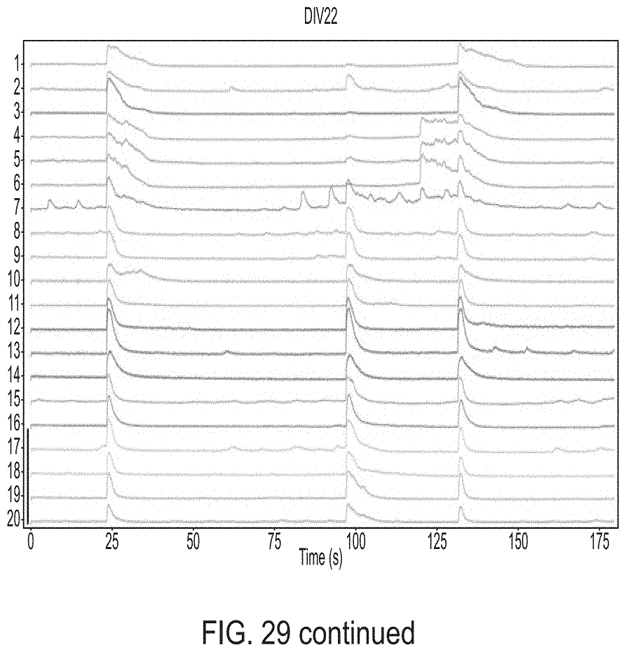

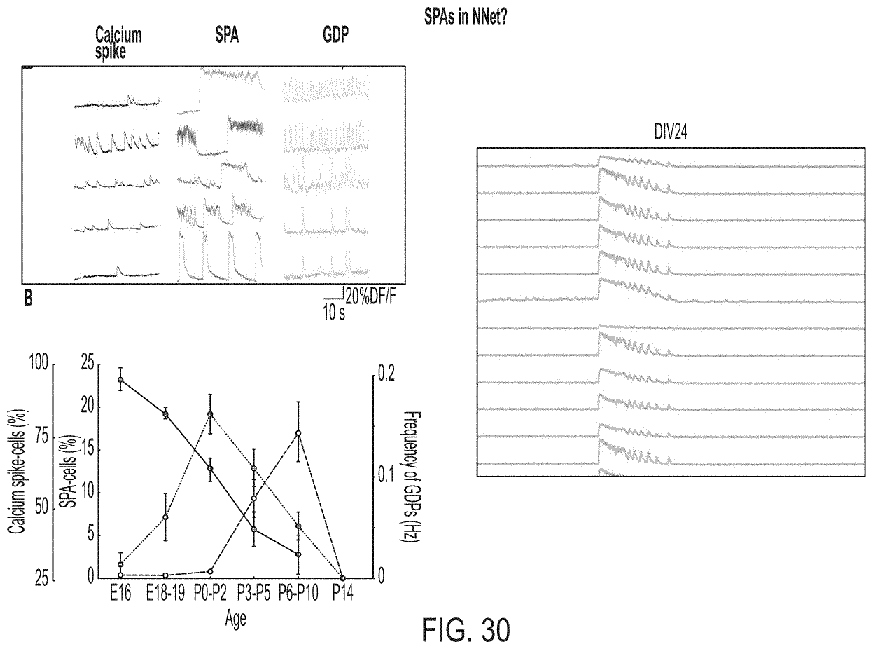

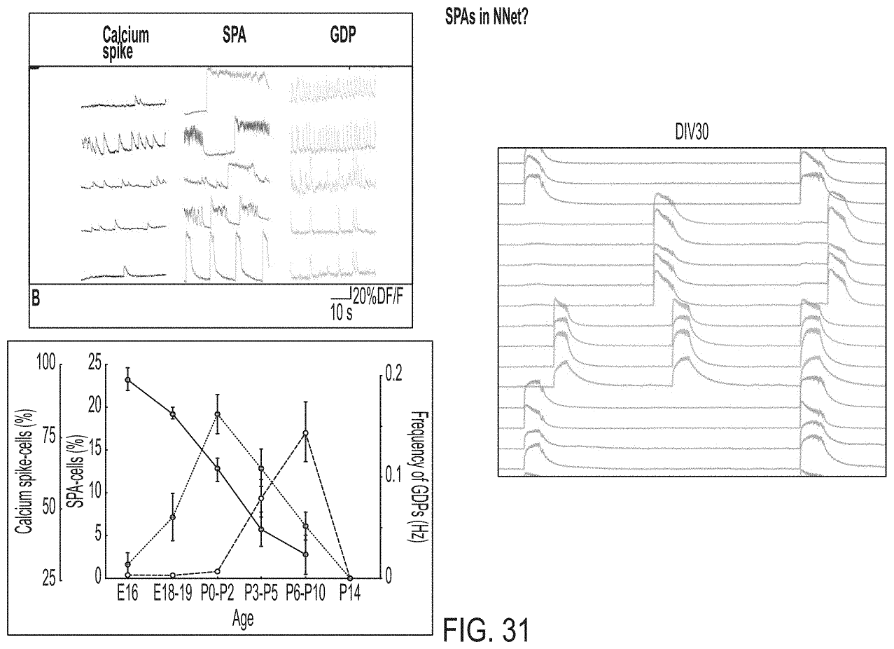

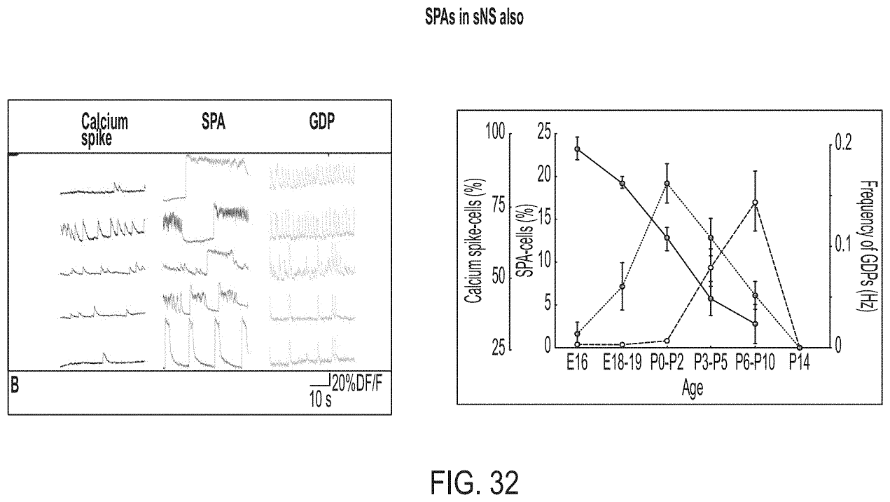

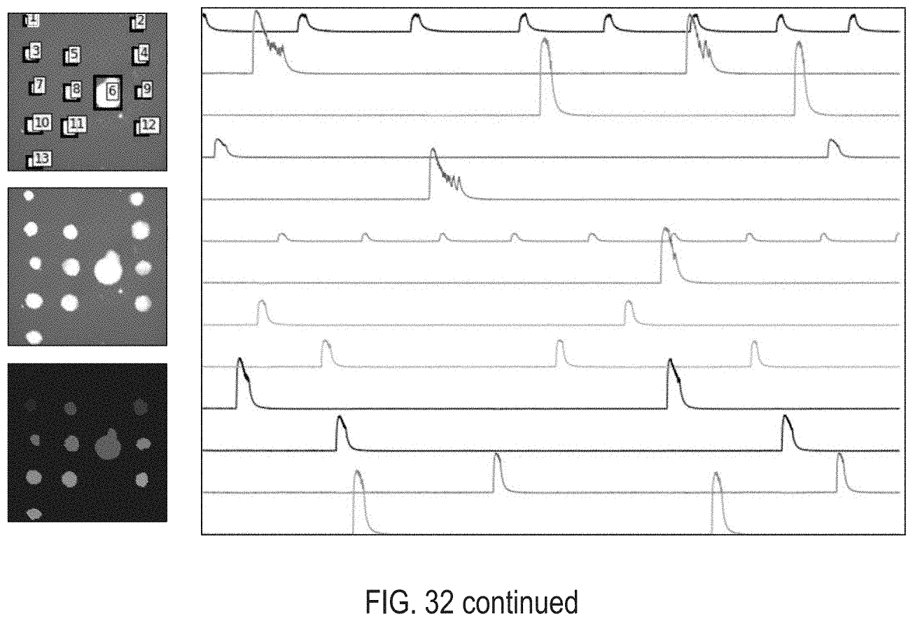

FIG. 25 to FIG. 33 show results of evaluating calcium spikes, Synchronous Plateau Assemblies (SPAs), and GDPs in NNets. Cells were loaded with fura 2-a.m. and the slice was imaged using multibeam two-photon excitation with a 20× objective. Acquisition rate was 100 ms/frame. Long-lasting calcium transients were visible in several cells. In FIGS. 26, the three images on the right show the first frame of a representative video.

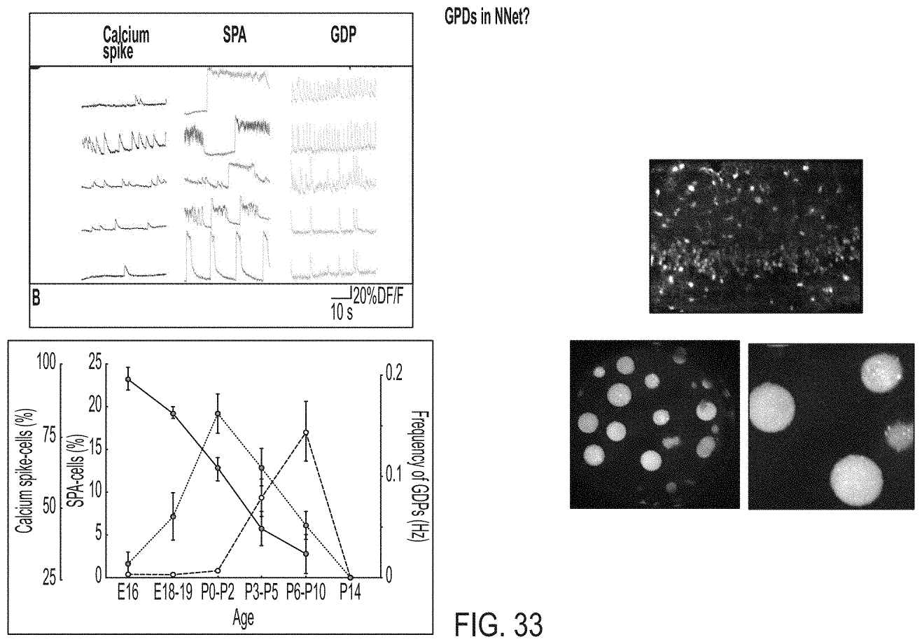

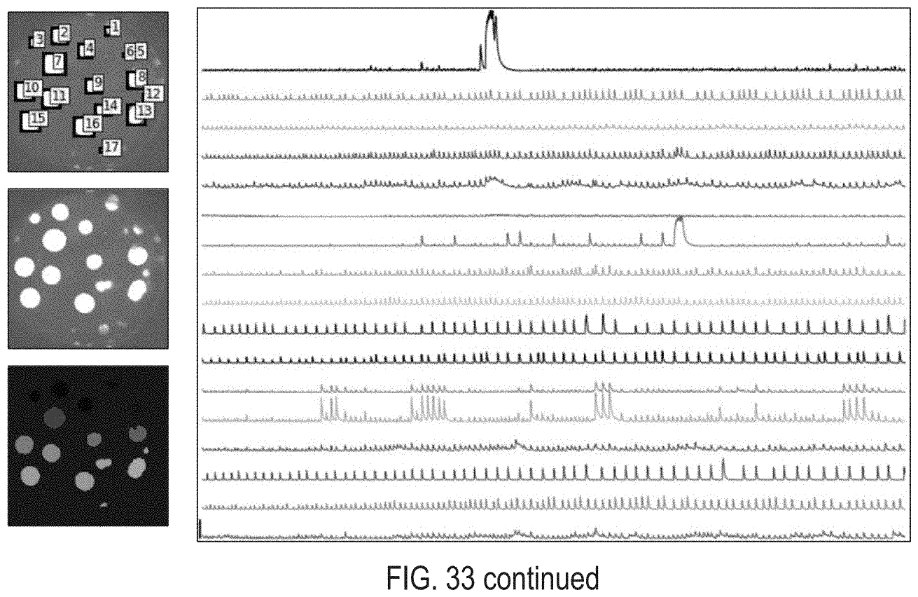

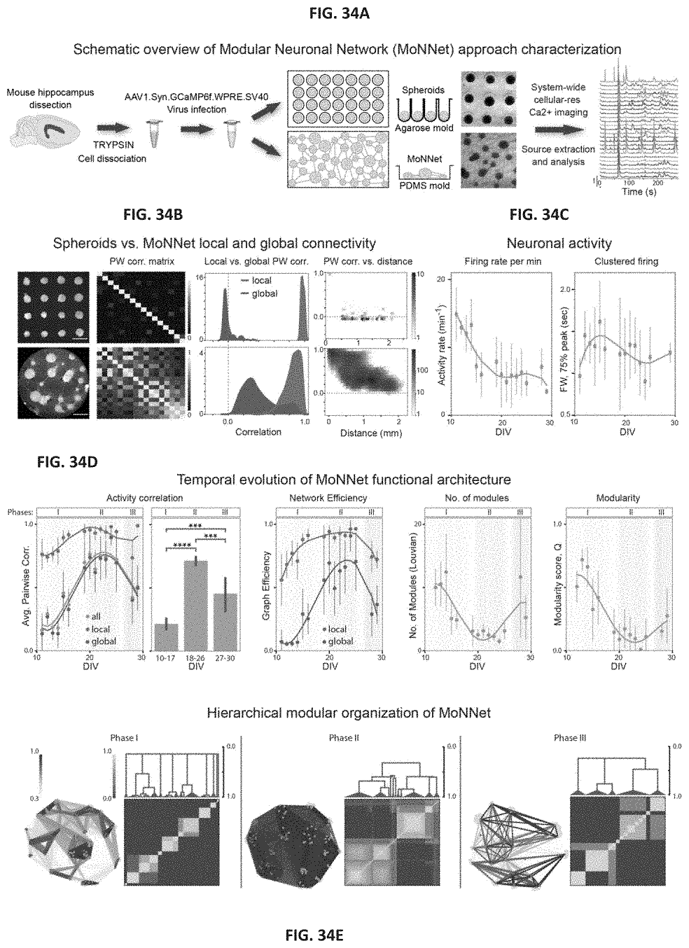

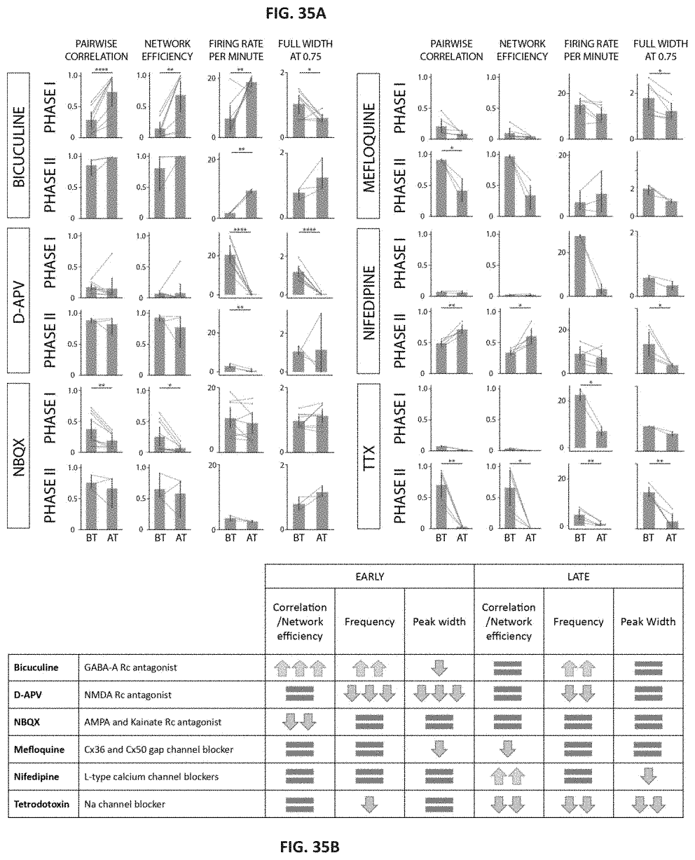

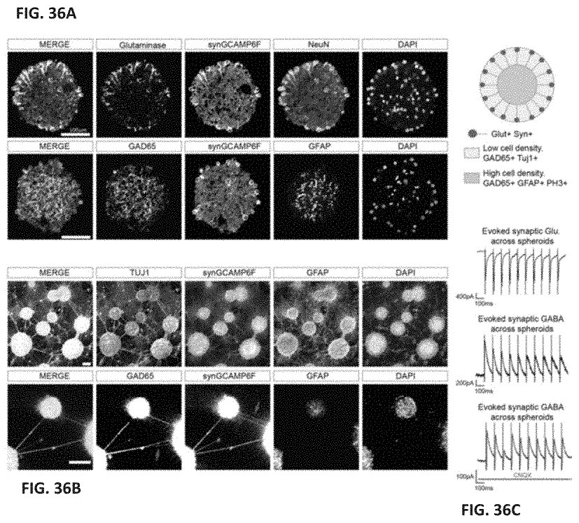

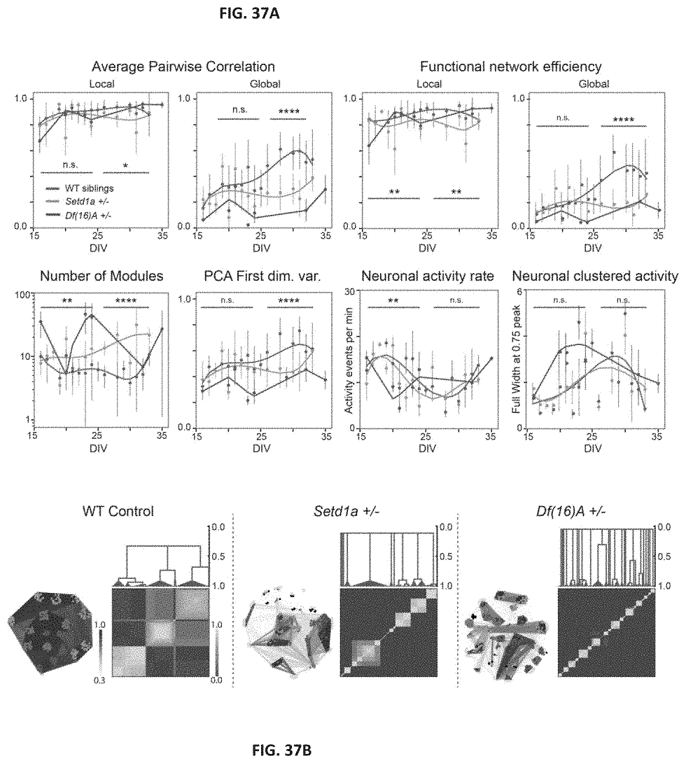

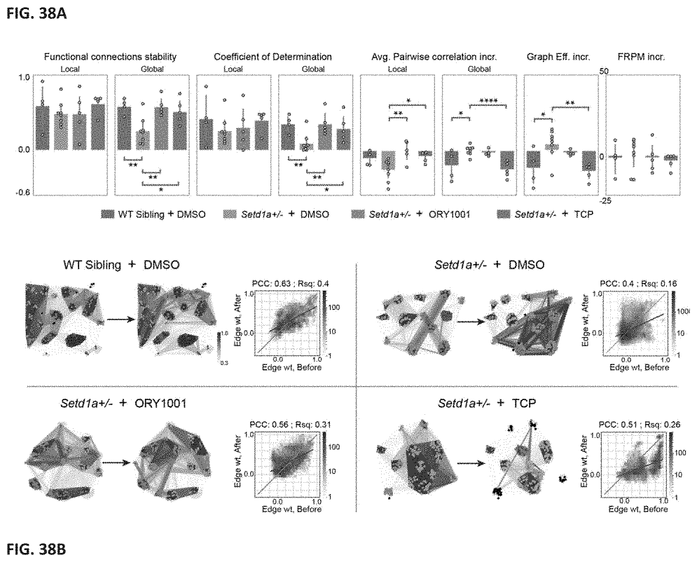

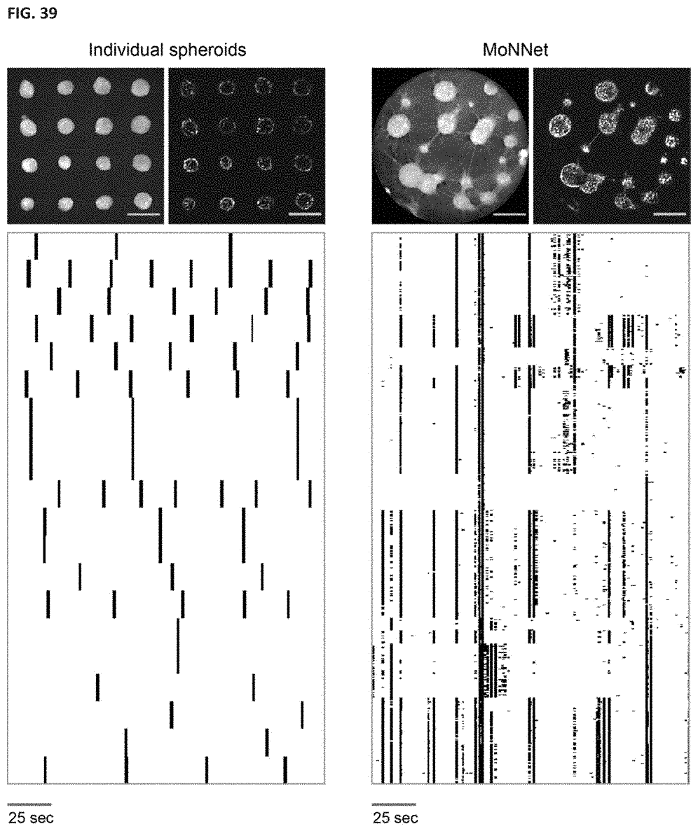

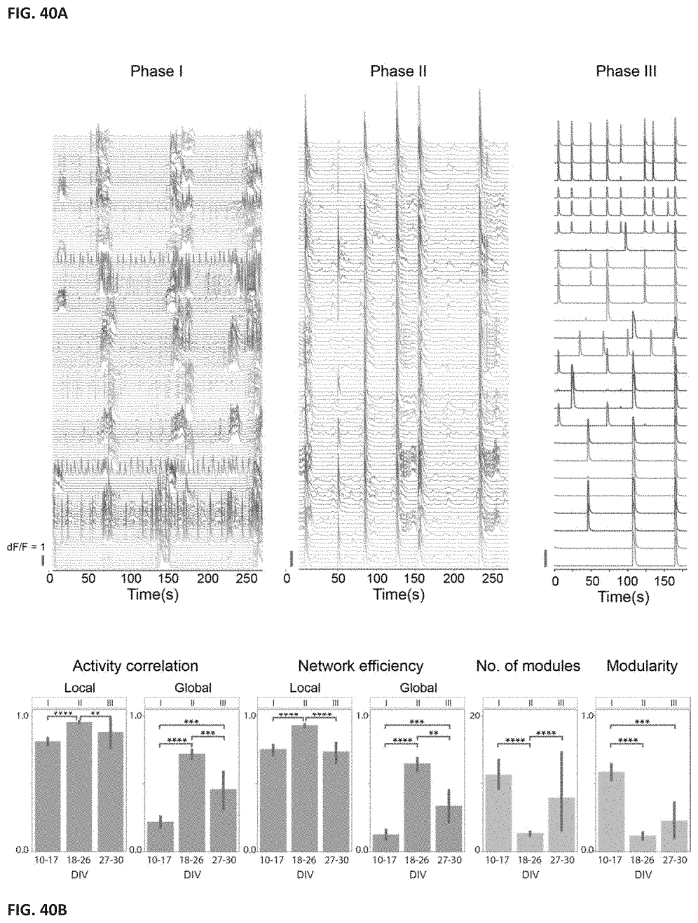

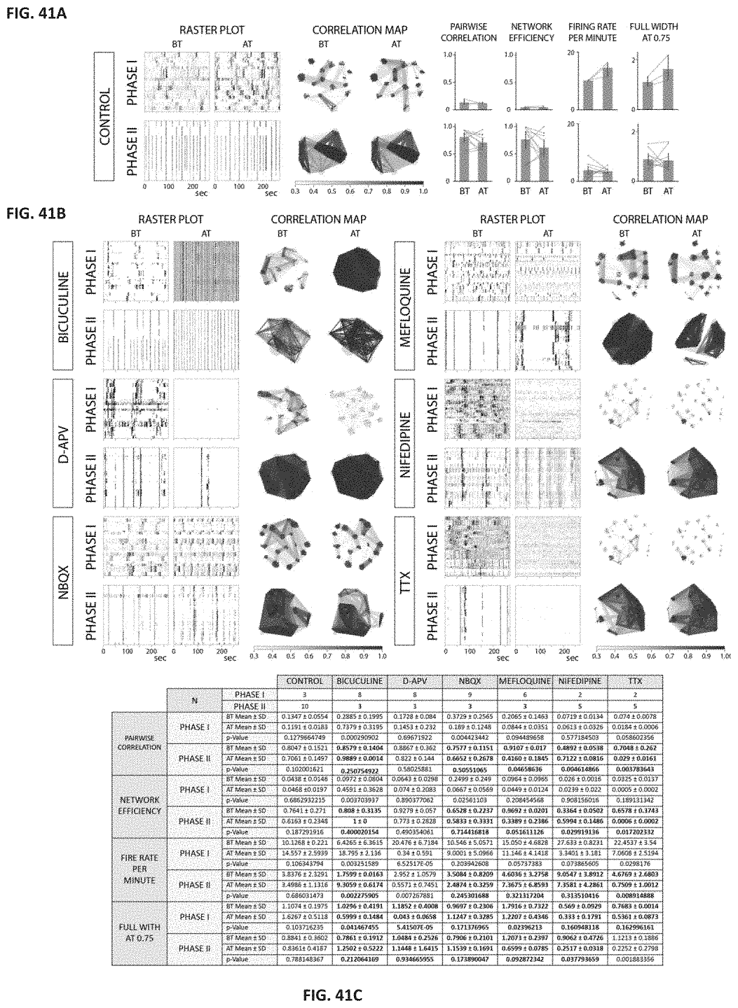

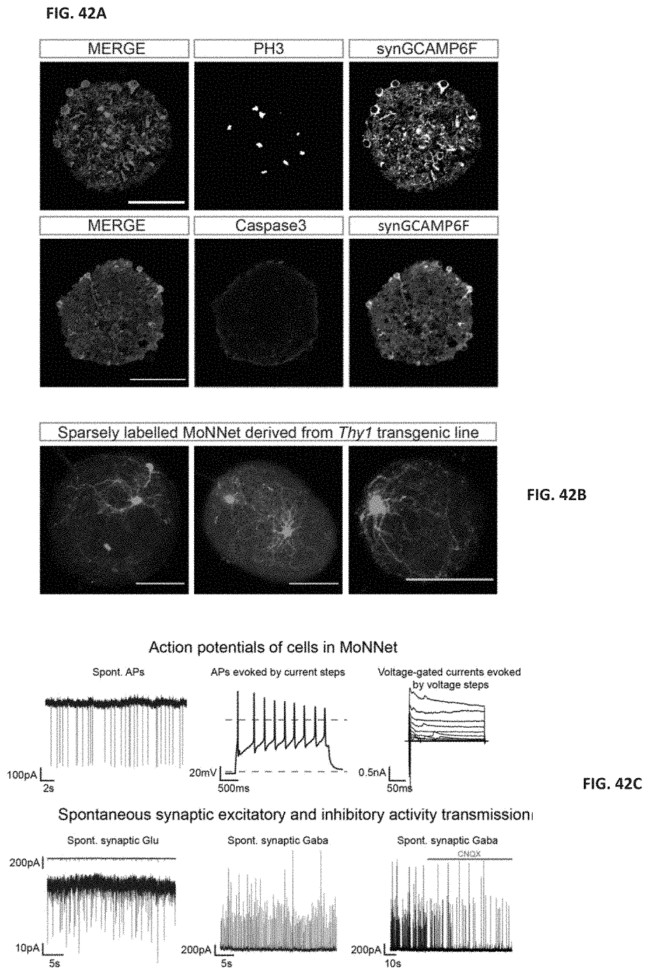

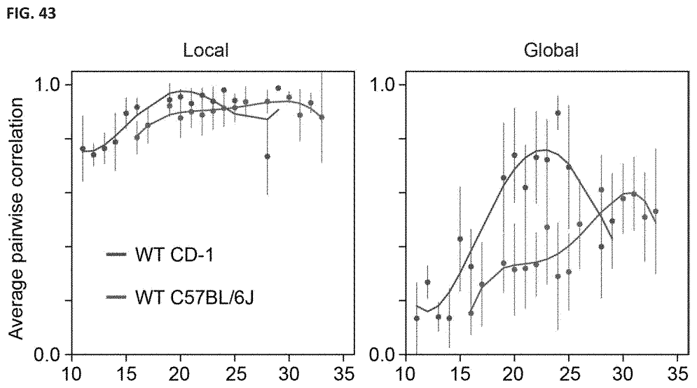

In FIG. 33, the three images on the right show the first frame of a representative video of spontaneous activity in the mouse CA1 hippocampal region in control conditions at the end of the first postnatal week (P6). Synchronous fast calcium transients are clearly visible. The following examples serve to illustrate certain aspects of the disclosure and should not be construed as limiting the claims. The contents of all references, pending patent applications and published patents, cited throughout this application are hereby expressly incorporated by reference.

### EXAMPLES

### Example 1: Self-Organized NNet

FIG. 1A is a schematic summary of methods for growing individual and network of neurospheres. Neuronal tissues are extracted from cortex/hippocampus of late stage embryos (E18 or early post-natal stages), followed by Trypsin treatment. Alternatively, human iPSC-derived neural progenitor cells can be used. The dissociated cells are infected with AAV1.Syn.GCaMP6f.WPRE.SV40 vector and plated in agarose-based micro-wells or in a flat PDMS mold (to yield NNet). Micrograph of an example NNet is shown. FIG. 1B shows single brain neurospheres exhibiting slow oscillatory activities. dF/F traces were extracted by overlaying datasets with a grid of super-pixels (16×16 microns). The traces are plotted separately in the graph, as identified by numbers 1 and 2. FIG. 1B shows activity of single and isolated neurospheres, while FIG. 1C shows that a NNet exhibit much more complex activity patterns. A NNet preparation was imaged for three consecutive days for 4 minutes durations (100 sec data shown for clarity). dF/F traces belonging to particular NNet node are plotted separately, as identified by the colored numbers.

### Example 2: Self-Organized NNet

FIG. 2 illustrates correlated neuronal activity (ensembles) in individual neurosphere vs. NNet preparations. Neurospheres activity profiles (dF/F) are shown from both individual (top) and NNet (bottom). Heat maps shows the pairwise correlations. As evident from the data, NNet activity is coordinated and synchronized, unlike in individual neurospheres.

### Example 3: Preparation NNet of Specific Configuration

FIG. 3 shows how NNet of a defined composition (i.e. of different types, marked by different colors) and connectivity patterns (and hence complexity) can be derived by first growing individual units in agarose or PDMS mold followed by their placement on a patterned mold made of PDMS or similar material. Neurospheres can establish inter-connections either passively, or activity when stimulated by axonal guidance directional cues such as Netrins. Netrins or similar axonal guidance molecules can also be used to control the directionality of the connections, when delivered in spatially restricted manner (e.g., using Netrin soaked microbeads).

### Example 4: Chronic Ketamine Treatment NNet Model of Schizophrenia Pathophysiology

NNet preparations were treated with ketamine (10 μM final concentration) every 24-hours. Calcium imaging was performed before adding ketamine and 2 days after. dF/F traces for all cells pre-/post-ketamine treatment are shown in FIG. 4. In FIG. 4A the micrograph on the left shows the NNets. Principal component analysis (PCA) was performed to determine clusters of co-active neurons (i.e. ensembles). All the cells belonging to the top three principal components are color-plotted in sample space, and the corresponding principal component traces are shown. As evident, identified ensembles before treatment were affected (marked by yellow arrows). Similarly, in FIG. 4B, dF/F traces and PC components are shown for a time control series. As evident, the spatial structure of ensembles remains intact, even though the temporal profiles have changed, similar to observations in mice. These experiments provide strong support for the feasibility of using NNet for modelling of schizophrenia pathophysiology.

### Example 5: Loss of Correlation (Neuronal Ensembles) in Chronic Ketamine Treatment NNet Model of Schizophrenia Pathophysiology

NNet preparations were treated with ketamine (10 μM final concentration) or saline control every 24-hours. Calcium imaging was performed before adding ketamine/saline control and 2 days after. The resulting dataset in FIG. 5 demonstrates that the correlation among neurosphere units of NNet is decreased in ketamine treated preparations. The graphs show dF/F traces of different neurospheres from the same network. On the right in FIG. 5, heat maps show pairwise-correlation among neurospheres. As evident, NNet activity correlation increases with time in control conditions, but decreases in the ketamine model. These results are consistent with data published from Schizophrenia studies in mice, which indicates that the NNet approach is useful for modeling complex brain disorders.

### Example 6: Modeling Epileptic Seizures with NNet

4-AP, a commonly used seizure inducing compound, was injected in a specific neurosphere of an NNet preparation. In FIG. 6, the first graph on the top shows the typical high seizure peaks produced by 4-AP local injection. As shown in the magnified view (top row, third) these peaks are maintained during the time. However, the addition of picrotoxin (GABA receptor antagonist) reduces the inhibition resulting in increased frequency of number of events. In FIG. 6 the colored maps (bottom) show the spatial progression of the seizure waves in three different bouts (labeled as Epi. Events #1, #2, and #3). The directionality of seizure spread is maintained constant from bottom-left to top-right. However, when picrotoxin is added, the directionality becomes random (see bottom of FIG. 6). These results are consistent with the data published from Epilepsy studies on brain mice, which indicates that the NNet approach is useful for modeling complex brain disorders.

### Example 6: Drug Screening

In a typical drug screening experiment, the NNet cultures (as described elsewhere), either derived from brain cells taken from a disease model or differentiated from iPSC cells from a patient, will be exposed to a candidate drug molecule. In parallel, a control network derived as above will be exposed to the same procedure but without the drug molecule. In addition, optionally when available, the NNet cultures would be prepared from mammal 2 with no known diseases. Note that, with the accumulation of data, we may not always need to use sample from mammal 2 as we will have enough documentation of normal behavior of such preparation.

These cultures will be subjected to high-speed live imaging in regular interval (e.g. every 3 hours) for several days (e.g. 10 days) to capture the activities of all brain cells, and their evolution over time. At the end of the live recording experiments, these cultures will be fixed using chemical fixatives (e.g. paraformaldehyde), and will be stained with antibodies and other reagents (e.g., antisense RNA) to identify molecular identity/type of all neurons.

The live recording image datasets will be analyzed to extract activity patterns of individual neurons, which will be further quantified with a multitude of descriptors, including the activity pattern motifs, the development of local (i.e. within units) and global synchrony over time. These descriptors will be compared across the preparations to assess the level of recovery achieved by the tested candidate drug molecule.

Such process will be repeated for a large number of potential drug candidates, either manually or by an automated process, one implementation of which may utilize microfluidics based devices for automating the growing of the culture, and the delivery of specific drug candidates, while recording with a microscope device.

Note that all experiments will be performed under strict control of environment parameters including temperature and humidity.

### Example 7: Setd1a+/− and Df(16)A+/− MoNNet Models

Taking advantage of MoNNet properties, effective in vitro models of SCZ-associated network pathophysiology (SCZ-MoNNets) were developed. SCZ is characterized by psychosis, cognitive dysfunction and a broad spectrum of complex behavioral abnormalities (Clementz et al., 2016). Several recent studies in animal models have investigated the underlying circuit pathophysiology, identifying deficits in activity synchrony and degraded ensembles (Fenelon et al., 2013; Hamm et al., 2017; Hamm et al., 2020; Marissal et al., 2018; Sigurdsson et al., 2010; Zaremba et al., 2017). Therefore, the MoNNet approach provides a well suited system to develop quantitative in vitro models of SCZ-like network function pathophysiology. We developed and characterized MoNNet preparations (SCZ-MoNNets) from two well-studied genetic models—Setd1a+/− and Df(16)A+/−, which recapitulate SCZ-related cognitive and circuitry pathophysiology (Fenelon et al., 2013; Mukai et al., 2019). An exhaustive comparative characterization of SCZ-MoNNets revealed degradation of modules/ensembles formation, altered global network synchrony, and much reduced inter-modular functional connections, in remarkable similarity to in vivo observations. The lower-level deviations in molecular, cellular and synaptic pathways underlying the observed alterations in network states remain to be determined and may be different for each mutation. Nevertheless it is worth noting that both mutations lead to alterations in terminal axonal growth, excitability and short term plasticity while the 22q11.2 deletion has been shown to have an impact on inhibitory neuron function (Fenelon et al., 2013; Mukai et al., 2019; Mukai et al., 2015; Mukherjee et al., 2019; Sun et al., 2018). Furthermore, we tested the applicability of SCZ-MoNNets as potential drug screening system by characterizing effects of antagonists of LSD1 methylase activity(ORY-1001 and TCP), which were recently shown to be effective in partial rescue of cellular and behavioral defects (Mukai et al., 2019). We found that even 2-day treatments of Setd1a+/− MoNNets with these compounds was sufficient to cause partial rescue of ensembles stability and network synchrony. These results strongly highlight the potential of MoNNets in developing in vitro models of complex brain disorders for understanding the underlying circuit pathophysiology as well as for establishing high-throughput screens for potential drug candidates.

### Animals and Genotyping

All animal handling and experimentations were done according to US National Institutes of Health guidelines and approved by the Institutional Animal Care and Use Committees(IACUC) of Columbia University. Pregnant wild-type CD-1 mice were purchased from Charles River laboratories at E11.5, and maintained in a temperature-controlled environment on a 12-h light-dark cycle, with ad libitum food and water until the experiment day. Df(16)A^{+/-} and Setd1a^{+/-} mice were obtained from an in-house colony. Briefly, Df(16)A^{+/-} mice (RRID: MGI_3802827) were generated on a CS7BL/6J background as described previously (Stark et al., 2008). Setd1a^{m1a(EUCOMM)Wtsi} mice (referred to as Setd1a⁺¹) were obtained from EMMA (https://www.infrafrontier.eu/search) and backcrossed in the C57BL/6J (The Jackson Laboratory, Bar Harbor, Me.) background as described previously (Mukai et al., 2019). Wild-type littermates from Setd1a^{+/-} crosses to WT mice were used as controls. The embryos were genotyped by PCR analysis of tail genomic DNA. For Setd1a crosses, following primer combinations were used:

### Setd1a_F (5′-GGTTATTGATCTGGGCAGGC-3′) and

Setd1a_R (5′-TGACCTGTTTTTCAAGCCCTC-3′); Setd1a_F and CAS_R1_Term (5′-TCGTGGTATCGTTATGCGCC-3′), to amplify the wild-type and mutant alleles with 546 and 241 base pairs expected band sizes respectively. For Df(16)A^{+/-} crosses, Df(16)A_F (5′-ATTCCCCATGGACTAATTATGGACAGG-3′) and Df(16)A_R (5′-GGTATCTCCATAAGACAGAATGCTATGC-3′) were used to amplify a 829 bp band for mutant allele, and Control_F (5′-CTAGGCCACAGAATTGAAAGATCT-3′) and Control_R (5′-GTAGGTGGAAATTCTAGCATCATCC-3′) were used to amplify a 324 bp IL-2 internal control band. The PCR program comprised denaturation at 94° C. for 5 min, followed by 35 cycles of 30 sec at 94° C., 30 sec at 58° C., and 45 sec at 72° C., and a final extension step at 72° C. for 5 min. For Thy1-eYFP transgenic lines experiments, B6.Cg-Tg(Thy1-YFP)HJrs/J strain mice were procured from Jackson Laboratory (Ref. 003782), and bred in Columbia University animal facilities. Embryos were genotyped by PCR analysis of the tail genomic DNA using 5′ CGGTGGTGCAGATGAACTT 3′ and 5′ ACAGACACACACCCAGGACA 3′ primers. PCR program comprised denaturation at 94° C. for 2 min, followed by 35 cycles of 20 sec at 94° C., 15 sec at 65° C., and 10 sec at 68° C., and a final extension step at 72° C. for 2 min.

### 3D Cell Culture Preparation

Hippocampal neuronal cultures were generated from E17 to E18 embryos by building upon standard cell culture techniques, as follows. Pregnant mice were anesthetized under isoflurane, and euthanized by cervical dislocation. Hippocampus were dissected in Hibernate E (Gibco) iced cold media, and incubated in 0.25% Trypsin-EDTA (Gibco) at 37° C. for 30 mM., followed by 5 mM DNAse I (1 μg/ml; Sigma) incubation at room temperature. Mechanical dissociation of dissected hippocampus was performed by repeated pipetting with a fire-polished glass Pasteur pipet until a homogenous cell suspension was obtained. Cell viability was determined by Trypan Blue exclusion assay. Cell solution was then centrifuged at 150 g for 10 mM, and the supernatant was removed. Resulting cell pellet was resuspended in the culture media containing Neurobasal media, 2% B27, 0.5 mM Glutamate and 1% Penicillin/Streptomycin (Gibco). Cells were infected with AAV1.Syn.GCaMP6f.WPRE.SV40 virus ((Chen et al., 2013); Pennsylvania Vector Core, Cat #:AV-1-PV2822). For single spheroid cultures, 2% agarose 96 wells (400 μm diameter) micro-molds were created by using custom casts fabricated by 3D printing (UV-resin; Formlabs 3D printer). The agarose micro molds were equilibrated in the culture medium over a 24 h period prior to plating. Approximately 10⁵ cells were seeded in the agarose micro mold. The cells were allowed to settle for 15 mM., followed by addition of 2 mL culture media. For the MoNNet, we generated custom polydimethylsiloxane (PDMS) molds, containing four 28 mm diameter wells, by polymerizing overnight (90° C.) in custom casts fabricated with acrylonitrile butadiene styrene (ABS; Ultimaker 2+). Approximately 2×10⁴ cells were seeded in each of the four wells in the PDMS mold. All cell cultures were kept in an incubator at 37° C. and 5% CO₂.

### Pharmacology

Synaptic and ion channel inhibition experiments were performed with bicuculline (10 μM, Cat #14340, Sigma), NBQX (10 μM, Cat #N183, Sigma), D-APV (40 μM, Cat #A8054, Sigma), Nifedipine (10 μM, Cat #N7634, Sigma), TTX (1 μM, Cat #1078, Tocris) and Mefloquine (25 μM, Cat #M2319, Sigma). The rescue experiments were performed with Tranylcypromine (TCP, 600 nM, Sigma, P8511) and ORY-1001 (0.6-0.9 nM, Cayman Chemical, 19136). All the compounds were added in the culture media as described in results.

### Time-Lapse Imaging

Imaging was performed using a wide-field fluorescence microscope (Leica M165FC) equipped with a long-pass GFP filter set (Leica filter set ET GFP M205FA/M165FC), 1.6× Plan Apo objective, 3.2× zoom and sCMOS camera (Hamamatsu ORCA-Flash 4.0). Time-lapse videos were recorded at 30 Hz for ˜4.5 min at 37° C.

### Immunohistochemistry

Embryos were fixed for 30 min at 4° C. in 4% PFA, and immunostaining was performed on either vibratome sections (50 μm) or whole mount MoNNets. For vibratome section staining, the sections were washed in PBS+0.1% Triton X-100 and incubation in blocking solution (PBS+0.1% Triton X-100+1% BSA) for 40 min, followed by overnight incubation with primary antibody (in blocking solution) at room temperature. Then sections were washed with PBS-0.1% Triton X-100, followed by incubation in secondary antibodies (1:500 dilution, in blocking solution) for 2 hours. Finally, sections were washed with PBS-0.1% Triton X-100 and mounted on slides for Confocal imaging. The following antibodies were used: Rabbit α-Glutaminase (1:500, Cat #Gltn-Rb-Af340, Frontier Institute Co. LTD.), mouse α-NeuN, clone A60 (1:50, Cat #MAB377, Millipore), rat α-GAD65 (1:1000, GAD-6, DSHB), rabbit α-GFAP (1:500, Cat #Z0334, Dako), rat α-phospho-Histone H3 (1:200, Cat #h9908, Sigma), mouse α-TUJ1 (1:200, Cat #801201, BioLegend) and rabbit α-Caspase3(1:500, Cat #559565, BD Pharmingen). Alexa Fluor 568-, and 647-conjugated secondary antibodies were obtained from Invitrogen. For whole mount MoNNets, the antibody blocking solution consisted of PBS+0.3% Triton X-100+0.5% BSA. Primary antibodies were incubated for 3 days at 4° C. while the secondary antibody for 4 h at room temperature. DAPI was used at 1 μg/ml, and used with the secondary antibody. Images were acquired using confocal microscope (Zeiss, LSM700) using 10×(EC Plan-Neofluar 10×/0.30 M27) and 20×(HC Plan Apochromat, NA 0.70).

### Electrophysiology

Whole cell recordings were performed as described previously (Crabtree et al., 2016; Crabtree et al., 2017), using borosilicate glass pipettes (initial resistance 3-5.5 MS2) which were filled with an intracellular solution containing: K methanesulfonate 125 mM, NaCl 10 mM, CaCl₂) 1 mM, MgCl₂ 1 mM, HEPES 10 mM, EGTA 0.1 mM, MgATP 5 mM, NaGTP 0.5 mM. The pH was adjusted to 7.2 with KOH. Spontaneous synaptic events were assessed at −70 mV (presumptive glutamatergic) and at 0 mV (presumptive GABAergic). Excitatory and inhibitory evoked synaptic responses were assessed as follows. Evoked synaptic responses across spheroid units were elicited with electric stimulation applied with a concentric bipolar stimulating electrode (tip diameter 0.125 mm, FHC, Bowdinham, Me.) positioned on a spheroid >250 μm away from the spheroid containing the recorded neuron. The stimulus was set to 10V, a duration of 100 μs and applied at 10 Hz. After achieving whole cell configuration, neurons were initially held in voltage-clamp at −70 mV to assess evoked excitatory synaptic responses, followed by washing-in of CNQX (10 μM) with ACSF (artificial cerebral spinal fluid) perfusion which completely ablated evoked excitatory synaptic responses at −70 mV, confirming these responses as mediated by glutamate. Neurons were then held in voltage-clamp at 0 mV to assess evoked inhibitory synaptic responses. For assessing neuronal excitability in current-step, action potential firing was recorded in response to incremental (20 pA steps) depolarizing current injections (500 ms duration). Bridge balance of series resistance was employed and recordings with series resistance >20 MΩ were rejected. For current-step assays, resting membrane potential was adjusted to ˜−70 mV by injection of a small standing current. For voltage steps, cells were held at −70 mV, and 10 mV steps were applied ranging from −100 mV to 50 mV. Series resistance-related errors were partially corrected by using 70% prediction and 70% series resistance compensation.

### Data Analysis

Motion Artifacts Correction.

All Ca²⁺ imaging datasets were first manually screened for any motion artefacts during the recordings. Datasets having motion artifacts were corrected by using the MOCO algorithm implemented as plugin (Dubbs et al., 2016) in ImageJ/Fiji (Schindelin et al., 2012; Schneider et al., 2012).

Segmentation and Aggregated Signal of Spheroid Units.

A custom watershed segmentation pipeline was implemented in Python by using scikit-image module (van der Walt et al., 2014). Maximum projection images (along the time axis) were used as inputs for segmenting the individual spheroid units. Resulting segmentation labels were manually inspected for any errors, which were corrected by adjusting the upper and lower thresholds, and the object size filter. Aggregated signal traces of each spheroid unit were calculated as average values in labels overlaid on Ca²⁺ imaging background-subtracted time-frames. Note that the background of each time-frame was estimated as the average pixel value outside the total segmentation mask. Aggregated signal was normalized by subtracting and dividing by the baseline values (estimated as the 8th percentile value in a sliding window of 500 frames).

Neuronal Activity Traces Extraction.

A custom pipeline in Python was used to localize activity sources by performing joint spatio-temporal deconvolution using constrained non-negative matrix factorization (CNMF) (Giovannucci et al., 2019; Pnevmatikakis et al., 2016; Zhou et al., 2018). Spurious non-spheroid sources were filtered by using the spheroids segmentation masks. For each identified unique spatial footprint, top 25% most probable pixels were used to calculate the average signal from background subtracted images. Signal traces were normalized (∂F/F) by subtracting and dividing by the baseline values, which was estimated as 8th percentile signal in a sliding window of 500 frames. A medium filter was applied to denoise the traces. Activation traces were temporarily deconvolved to infer spikes using OASIS method (Friedrich et al., 2017). Second order generative autoregression model was used.

Local and Global Average Pairwise Calculations.

Average pairwise activity correlation was calculated as average of Pearson's correlation coefficient in aF/F traces of all possible pairs of neurons in a sample. Local and global average pairwise correlation were calculated only from the pairs belonging to the same and the different spheroid units, respectively.

Firing Rate and Clustered Firing Duration.

Firing rate per minute was calculated from estimated spike trains. A threshold of 5 standard deviation was used to identify significant firing events for calculations. Average firing rate were calculated by averaging over all neuronal sources belonging to a MoNNet sample. Clustered activity duration was calculated as the full width at the level of 75% of the aF/F peak, as follows. For first identified firing event in the time-series, a forward iterative traversal was performed on aF/F time-series to identify the peak time-position, followed by forward and backward iterative traversal to calculate the full width at 75% of the peak level relative to the baseline. The procedure was repeated for the next spike position, not overlapping with the already analyzed peaks. Average value was calculated for all peaks identified in an individual neuron, which were then averaged over all neurons to yield the average clustered firing duration.

Local and Global Graph Efficiency Calculations.

In the first step, weighted graphs were generated by representing neurons as nodes, and the pairwise correlations between a pair of neurons as the weighted edge. A threshold of 0.8 was consistently applied on the edge weights to generate binary graphs, which were used to calculate the global and local graph efficiencies using the NetworkX python modules. Note that the trends looked similar for a range of weight thresholds.

Modularity and Co-Classification Analysis.

Louvain community detection algorithm (Blondel et al., 2008) was used to detect modules in the MoNNet weighted graphs (discussed above). Note that a lower threshold of 0.5 was used to remove the weaker edges from the weighted graphs for these calculations. Nodes co-classification (into the same modules) probabilities were calculated by using multiresolution modularity and consensus clustering (Jeub et al., 2018) analysis of binary graphs generated by consistently using an edge weight cutoff of 0.7. The event sampling ensembles for the co-classification analysis were generated from 10,000 partitions of modularity resolution parameter (y). Other parameters were set to their default values.

Activity Source Extractions from Before and after Treatment MoNNets.

Ca²⁺ imaging datasets before and after pharmacological treatments were first aligned using a custom registration pipeline based on SimpleITK python implementation (Lowekamp et al., 2013). Maximum projection (along time axis) images from after-treatment datasets were registered to the corresponding maximum projection images from before-treatment datasets. Translational transformations and Mattes' mutual information similarity matrix (Mattes et al., 2001) were used for performing the registration. Optimized translational parameters were then applied to the entire after-treatment image stacks, to yield completely aligned datasets, which were inspected manually to ensure of high cellular-resolution accuracy. Finally, before and after treatment stacks were concatenated to detect activity sources using the pipeline discussed above, followed by calculation of various measures discussed above.

Although in embodiments, a mold of agarose is described, it is understood that other types of hydrogel or other non-adhesive polymers may be substituted therefore to generate additional embodiments.

All references cited herein are incorporated by reference in their entirety. While the above disclosure has been described with reference to exemplary embodiments, those of ordinary skill in the art will understand that various changes in form and details may be made without departing from the spirit and scope of the present invention as defined by the following claims.

1.-2. (canceled)

3. A method of making a neurosphere network, comprising:
providing a plurality of neurospheres, optionally artificial neurospheres, on a surface of polysaccharide or of a silicone-based organic polymer, wherein the neurospheres are spaced apart on the surface by an average of 2, 5, 10, or 15 mm, and
growing axons among neurospheres under culture conditions to form a neurosphere network.

4.-12. (canceled)

13. The method of claim 3, wherein the neurospheres are spaced apart on the surface by a maximum of 2, 5, 10, or 15 mm.

14. The method of claim 3, wherein the neurospheres are spaced apart on the surface by a minimum of 2, 5, 10, or 15 mm.

15.-16. (canceled)

17. The method of claim 3, wherein the neurospheres are formed by culturing precursor cells.

18. The method of claim 17, wherein the precursor cells include iPSC-derived neuronal progenitors.

19.-22. (canceled)

23. A method for screening a compound for activity against a brain disorder, comprising:
(a) contacting the compound with a first neural network obtained by a method according to claim 3, wherein the first neural network originated from brain cells obtained from a first mammal or derived from stem cells or iPSC cells of a first mammal presenting a disorder or a biomarker indicative of the brain disorder,
(b) contacting the compound with a second neural network obtained by a method according to claim 3, wherein the second neural network originated from brain cells, or brain cells derived from stem cells or iPSC cells, of a second mammal of the same species as the first mammal but lacking the brain disorder or biomarker; and
(c) recording activity patterns, using calcium imaging or electrophysiology, over time from the first and second neural networks after the contacting steps, and analyzing the activity patterns to determine a difference in generated signal characteristic of the brain disorder.

24. The method of claim 23, further comprising (d) fixating the neural networks after live recording, and staining and visualizing the molecular identity of the cells for a biomarker.

25. The method of claim 23, wherein the biomarker comprises a gene mutation or a biologically active protein.

26. The method of claim 25, wherein the biomarker comprises a neurotransmitter-related protein or molecule, for example parvalbumin, somatostatin, glutamate, GABA, or dopamine.

27. The method of claim 23, wherein the biomarker comprises a transcription factor or an effector gene marker.

28. The method of claim 23, wherein the signal comprises one or more factors, preferably the patterns of activity of individual brain cells and their evolution over time, synchronization or correlation of activity of all brain cells or brain cells belonging to individual units over time, or the population level activity evolution over time, as quantified by dimensionality reduction or stable temporal sequences of activities.

29. The method of claim 28, wherein the dimensionality reduction is principal component analysis, independent component analysis, or tSNE, or the relationship of activity patterns to underlying molecular identity of brain cells.

30. The method of claim 23, wherein the brain disorder is schizophrenia, epilepsy, autism, Parkinson's disease, depression, or a neurodegenerative brain disorder such as Alzheimer's disease.

31. The method of claim 23, wherein the compound is a small molecule, polynucleotide, protein, or virus particle.

32. The method of claim 31, wherein the compound is a DNA based or RNA based active compound, or a biologically active protein such as an antibody.

33. The method of claim 23, wherein the first mammal and the second mammal are mouse or human.

34. The method of claim 23, wherein the first mammal and the second mammal are mouse or human, and the brain cells are derived from stem cells or iPSC cells from the first or second mammal.
